(19) United States
(12) Patent Application Publication
(10) Pub. No.: US 2022/0296610 A1
(43) Pub. Date: Sep. 22, 2022

(54) COMPOSITIONS AND METHODS FOR TREATMENT OF INFECTION AND NOVEL COSMECEUTICAL PREPARATIONS

(71) Applicant: Derm-Biome Pharmaceuticals Inc., Vancouver (CA)

(72) Inventor: Burkhard Jansen, La Jolla, CA (US)

(21) Appl. No.: 16/603,154

(22) PCT Filed: Apr. 5, 2018
(86) PCT No.: PCT/US2018/026259

§ 371 Date: Oct. 4, 2019

Related U.S. Application Data
(60) Provisional application No. 62/482,338, filed on Apr. 6, 2017.

(51) Int. Cl. A61K 31/57; A61Q 11/00; A61P 31/04; A61K 45/06; A61K 8/63; A61Q 19/10; A61P 17/06; A61P 17/00; A61P 17/10; A61P 27/02; A61P 17/02; A61Q 19/00; A61Q 1/00; A61Q 1/10

(52) U.S. Cl. CPC A61K 31/57; A61Q 11/00; A61P 31/04; A61K 45/06; A61K 8/63; A61Q 19/10; A61P 17/06; A61P 17/00; A61P 17/10; A61P 27/02; A61P 17/02; A61Q 19/00; A61Q 1/00; A61Q 1/10

(57) ABSTRACT

Disclosed are methods of treating or preventing infection in a mammal in need thereof, by administering the following compound, or related compounds, to a mammal, as well as cosmeceutical preparations and methods of using the same to influence the appearance and/or biological function of the skin by topical application of the preparations comprising the following compound, or related compounds:

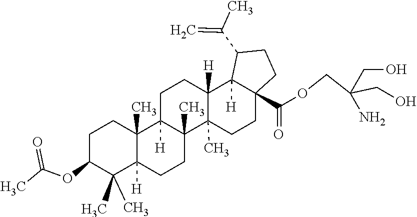

### FIELD

The present disclosure relates to compositions and methods for treatment of infection. More specifically, the disclosure relates to the use of the betulinic acid derivative 3-O-acetylbetulinic acid-2-amino-3-hydroxy-2-hydroxymethylpropyl ester (herein referred to as NVX-207), and related compounds, and to particular formulations comprising these compounds, as agents for the treatment of infection. Additionally, the present disclosure relates to cosmeceutical preparations comprising NVX-207, and related compounds, and to methods of influencing the appearance and/or biological function of the skin by topical application of these preparations.

### BACKGROUND

Betulin is a naturally occurring triterpene isolated by extraction from birch bark and other plant sources. Betulin is easily converted to betulinic acid, and both compounds exhibit various biological activities, including anti-malaria, anti-inflammatory, anti-HIV and anti-tumor effects (Sami et al., Eur. J. Pharm. Sci. 29:1-13, 2006). Antibacterial and antioxidant properties of betulinic acid and its derivatives has been discussed in a recent review (Yogeeswari P & Sriram D. Curr Med Chem. 2005 12:657-66). Betulinic acid induces apoptosis in various tumor cell lines, including melanoma cells. Anti-melanoma effects have been observed both in vitro and in vivo in SCID mouse xenotranplantation models (Pisha et al., Nature Med. 1:1046-51, 1995). Betulinic acid was also shown to induce apoptosis in normal and malignant skin cells, including keratinocytes and melanocytes (Galgon T et al., Exp Dermatol. 14:736-43, 2005). U.S. Pat. No. 7,312,205, the entire contents of which are incorporated herein by reference, describes various betulinic acid derivatives, and their use for treatment of HIV and various carcinomas, including melanomas, sarcomas, lymphomas, and squamous cell carcinomas.

Betulin can be present at concentrations of up to about 24% of the bark of white birch (Merck Index, twelfth edition, page 1236, 1996). Lupeol is a related compound also found in birch bark and in other plant sources. Lupeol is present at concentrations of about 1.5-3% of birch bark and at up to about 8.2% in Canavalia ensiformis, a plant widespread in the humid tropics of Asia, India, and Africa. Allobetulin is another triterpenoid found in birch bark. A typical pulp mill that processes birch produces enough bark waste to allow for the inexpensive isolation of significant quantities of these triterpenoids.

The plant, Centella asiatica, grows in Madagascar and around the Indian Ocean. Traditionally, this plant has been used for wound healing. See Chopra, R. et al., “Indigenous Drugs of India”, Dhur & Sons Pvt. Ltd. (1985), Calcutta. In Europe, a drug prepared from this plant is used for the treatment of ulcers and wounds. Centella asiatica is also suitable for cosmetic use, i.e., skin conditioning improvement, anti-cellulite effect, and improvements in skin color. See Adolphe, M. et al., Int. J. Cosmetic Soc., (1984), 6, pp. 55-58. Centella asiatica contains asiatic acid, madecassic acid, asiaticoside and madecaside, all of which belong to the class of triterpenoids. It has been shown that the triterpene extract from Centella asiatica stimulated collagen synthesis in fibroblast monolayer cultures, and asiatic acid was found to be the major component responsible for collagen synthesis stimulation. (Maquart et al., Conn. Tissue Res., (1990), 24 pp. 107-120)

Extracts of birch bark containing a mixture of betulin, betulinic acid and other terpenoids have been used to treat bacterial infections (U.S. Pat. No. 6,689,767). Betulin and related compounds have also been shown to have anti-viral activity against herpes simplex virus (Carlson et al., U.S. Pat. No. 5,750,578).

U.S. Pat. No. 6,303,589 discloses fungicidally effective compositions containing at least one pentacyclic triterpene compound. Katere et al. describe the antimicrobial activity of pentacyclic triterpenes isolated from African Combretaceae (Phytochemistry 63, 81-88, 2003). Nick et al. disclose antibacterial triterpenoids from Dillenia papuana and their structure activity relationships (Phytochemistry 40:1691-1695, 1995); Suskamrarn et al. disclose Ceanothane- and Lupane-Type triterpenes with antiplasmodial and antimycobacterial activities from Ziziphus cambodiana (Chem. Pharm. Bull. 54:535-537, 2006); Horiuchi et al. discloses antimicrobial activity of oleanolic acid from Salvia officinalis and related compounds on vancomycin-resistant enterococci (VRE) (Biol. Pharm. Bull. 30:1147-1149, 2007).

Bacteria are very common pathogens of mammals, especially humans. Among the bacterial species that cause serious disease are the gram negative bacterium Escherichia coli and gram positive bacteria of the genus Staphylococcus. Staphylococcus aureus is the most serious pathogen of the Staphylococcus bacteria, and is estimated to cause 13% of the 2 million hospital infections each year, resulting in 80,000 deaths annually in the United States. Staphylococcal infections occur most commonly in persons weakened by poor health and/or immunodeficiency. Bacterial infections are often associated with localized or systemic inflammation.

Antibiotic resistance of bacteria is a growing problem. New agents active against resistant bacteria are needed. A need particularly exists for agents that will act against a range of species, including gram-negative and gram-positive species. Ideally, new agents would also be inexpensive to manufacture.

Rosacea is a common multifactorial skin condition that is estimated to affect over 45 million people worldwide. It affects mostly Caucasians of north-western European descent and has been nicknamed “curse of the Celts” by some. It begins as erythema (flushing and redness) on the central face and across the cheeks. As rosacea progresses, semi-permanent erythema, teleangiectasia (dilution of superficial blood vessels) and red domed papules (small bumps) as well as pustules and a red lobulated nose (rhinophyma) develop. Rosacea may also present as ocular rosacea with red, dry and irritated eyes and eyelids. Taken together, four subtypes of rosacea have been described. Vascular endothelial growth factors have been implicated in the pathogenesis of rosacea (Smith et al., British Journal of Opthalmology, 91:226-229, 2007). Often unsatisfactory treatment options include sun protection, laser treatment, topical tretinoin, azelaic acid gel, steroid creams as well as topical and systemic antibiotics. Currently there is no cure for rosacea. New and improved treatment options are therefore clearly needed (Wilkin et al., J. A. Acad. Dermatol. 46:584-587, 2002). Betulinic acid and derivatives thereof, due their antibacterial, anti-inflammatory and anti-angiogenic effects, are therefore candidates especially well suited for local treatment of this disease. Anti-angiogenic effects of betulinic acid and derivatives are well known from the scientific literature (Mukherjee et al. Bioorg Med Chem Lett. 14:3169-72, 2004; Mukherjee et al. Bioorg Med Chem Lett. 14:2181-84, 2004).

One of the major obstacles for drug development and clinical application of betulinic acid and its derivatives is their extremely low solubility in polar solvents such as ethanol or in aqueous solutions. Most small animal models published to date utilize betulinic acid/betulinic acid derivative pharmaceutical formulations which are not well-suited for clinical use. Until now, betulinic acid or its derivatives have not been successfully used as mono-substances in the treatment of rosacea, bacterial infections or other skin or mucosal diseases. Betulinic acid and structural relatives containing extracts that are topically applied from the birch bark contain several potentially active structurally related as well as unrelated compounds.

Thus, there is a need for compositions and methods for effectively treating infections, which do not have significant side effects, and which have sufficient solubility in polar solvents such as ethanol and DMSO or in aqueous solutions to enable their use in conventional pharmaceutical formulations at sufficiently high effective concentrations.

Published PCT Application No. WO/2005/011717 entitled “USE OF BETULINIC ACID OR AN ASSAYED PLANT EXTRACT IN BETULINIC ACID INDIVIDUALLY OR ASSOCIATED FOR COSMETIC, NEUTRACEUTICAL, VETERINARY AND PHARMACEUTICAL USE” describes the use of a total extract or a triterpene-enriched extract of leguminoses such as Psophocarpus tetragonolobus and related species as antiviral, anti-inflammatory and anti-carcinogenic agents, and also the cosmetic, dermatological or pharmaceutical use of such compositions.

Betula alba birch bark extracts containing high levels of pentacyclic triterpenes (betulin and betulinic acid) are known for their ability to actively inhibit the enzyme elastase to prevent/correct the loss of elastic fibers responsible for skin suppleness. They stimulate collagen synthesis and inhibit inflammatory processes, inhibit melanogenesis to achieve skin lightening and improve skin tone and clarity.

Betulin has also been shown to bind melanocortin receptors and as a consequence thereof antagonize MSH induced cAMP generation in melanoma cells (Muceniece et al., Cell Biochemistry and Function 25, 591-59, Published Online: 2 Jul. 2007). These observations suggest an anti-pigmentary mechanism for betulin and betulin-like compounds, because of the well described involvement of melanocortin receptors in this process (Böhm et al., J. Invest. Dermatol. 2006 September; 126(9):1966-75.)

The US Federal Food, Drug and Cosmetic Act (the Act or FFDCA) defines cosmetics as “articles intended to be rubbed, poured, sprinkled, or sprayed on, introduced into, or otherwise applied to the human body for cleansing, beautifying, promoting attractiveness, or altering the appearance.” Among the products included in this definition are eye and facial makeup, skin moisturizers, lipsticks, shampoos, hair colors, sunscreens, deodorants, as well as any material intended for use as a cosmetic product (FDA/CFSAN, “Is it a Cosmetic, a Drug or Both (or is it Soap?”). U.S. Food and Drug Administration, Center for Food Safety & Applied Nutrition, Office of Cosmetics and Colors, Fact Sheet, 2002).

Cosmeceuticals are topical cosmetic-pharmaceutical “hybrids” which influence the biological function of the skin, and/or enhance the beauty of the skin or hair, via one or more active ingredient present in a cosmetic that provide a health-related function or benefit. These compositions may improve the appearance, functioning and/or texture of the skin by inhibiting the harmful effects of free radicals, thus improving the appearance of the skin by reducing the number and severity of wrinkles, reducing skin damage or reducing inflammation. Active ingredients often found in cosmeceuticals include antioxidants, antibacterial agents, exfoliants, UV-absorbing compounds, anti-aging compounds, anti-inflammatory agents and skin conditioning agents. Thus, cosmeceuticals can be considered a link between personal care products and pharmaceuticals. The use of cosmeceuticals has risen dramatically in recent years, and they are a fast growing segment of the personal care industry.

There is a constant need for novel cosmeceutical compositions, and for methods of improving the appearance and health of the skin. The present disclosure provides such cosmeceuticals.

### SUMMARY

The present disclosure provides a method of treating and/or preventing microbial infections (e.g., infections caused by bacteria, fungi, protozoa or viruses). This includes both topical and systemic administration of the compounds described herein for the treatment and/or prevention of infection, including skin conditions such as psoriasis, rosacea, and acne, in a mammal in need thereof.

The method comprises identifying a mammal in need of such treatment, and administering an effective amount of a compound having the following formula, a related compound, or a pharmaceutically acceptable salt thereof, to the mammal:

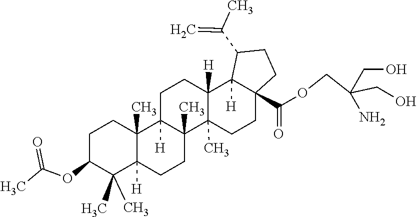

In one embodiment, the related compound has the following general formula:

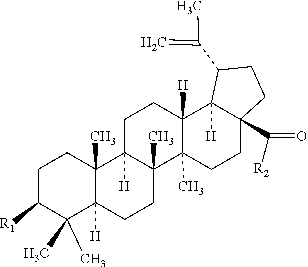

wherein R₁ represents a hydroxy group, an amino group, a protected hydroxy group or a protected amino group,

### and R₂=

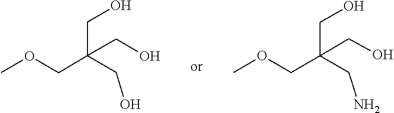

In another embodiment, R1 represents a hydroxy group, an amino group or one of the following protected hydroxy or amino groups:

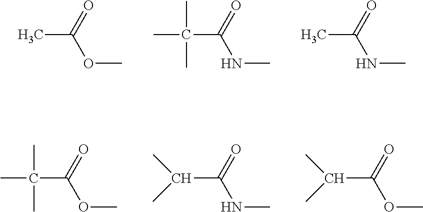

and R₂ is as defined above.

In one embodiment, the compound is administered topically as a lotion, ointment, gel, cream or paste. In other embodiments, the compound is administered systemically through oral administration or injection. In another embodiment, the compound is administered in a transdermal delivery system. The compound may be administered to the oral cavity. In certain embodiments, the compound is contained within a mouthwash, oral gel, oral spray or toothpaste. The compounds described herein are useful, for example, in the treatment of infection in mammals. In one embodiment, the mammal is a human.

The present disclosure also provides a pharmaceutical formulation comprising the following compound, a related compound, or a pharmaceutically acceptable salt thereof; ethanol and/or DMSO in an amount from 0.1-70%)v/v); and a nonionic detergent in an amount from 0.1-70% (v/v):

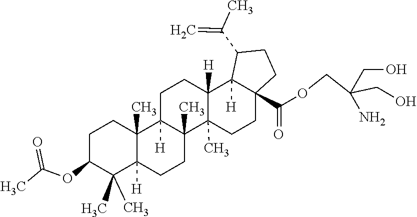

In one embodiment, the nonionic detergent is Cremophor EL® (polyethoxylated castor oil), Tween® 80 (polyoxyethylen-sorbitan-monooleate), Tween® 40 (polyoxyethylene (20)-sorbitan-monopalmitate), Triton® X-100 (polyethylenglycol-[4-(1,1,3,3-tetramethylbutyl)phenyl]-ether), Span® 20 (sorbitan monolaurate) or Span® 85 (sorbitan trioleate. In another embodiment, the ethanol and/or DMSO is present in an amount of 20% (v/v). In yet another embodiment, the nonionic detergent is present in an amount of 20% (v/v).

The present disclosure also provides a pharmaceutical formulation comprising the following compounds, or pharmaceutically acceptable salts thereof:

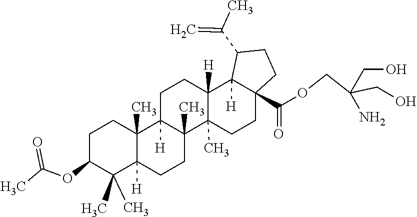

In formulations involving ethanol and/or DMSO or chemically similar solvents in an amount from 0.1-70% (v/v); and a nonionic detergent in an amount from 0.1-70% (v/v).

In one embodiment, the nonionic detergent is Cremophor EL® (polyethoxylated castor oil), Tween® 80 (polyoxyethylen-sorbitan-monooleate), Tween® 40 (polyoxyethylene (20)-sorbitan-monopalmitate), Triton® X-100 (polyethylenglycol-[4-(1,1,3,3-tetramethylbutyl)phenyl]-ether), Span 20 (sorbitan monolaurate) and Span 85 (sorbitan trioleate). In another embodiment, the ethanol and/or DMSO are present in an amount of 20% (v/v). In another embodiment, the nonionic detergent is present in an amount of 20% (v/v).

The present disclosure also provides a method of treating or preventing an oral infection in a mammal in need thereof, comprising identifying a mammal in need of such treatment, and administering to the oral mucosa of said mammal an effective antibacterial amount of a compound having the following formula, a related compound, or pharmaceutically acceptable salts thereof:

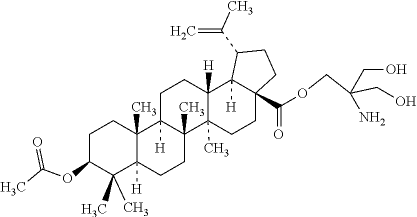

In one embodiment, the related compound has the following general formula:

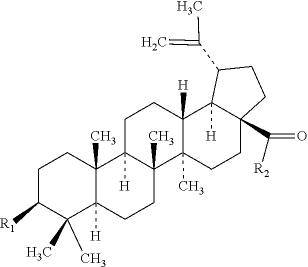

wherein R₁ represents a hydroxy group, an amino group, a protected hydroxy group or a protected amino group,

### and R₂=

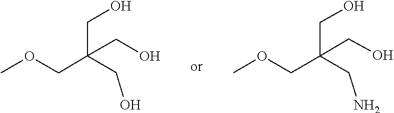

In another embodiment, R₁ represents a hydroxy group, an amino group or one of the following protected hydroxy or amino groups:

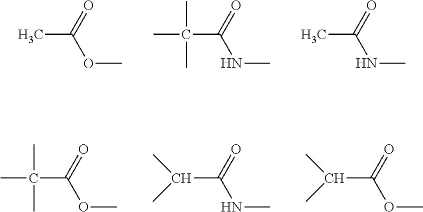

and R₂ is as defined above.

In one embodiment, the mammal is a human. In another embodiment, the oral mucosa is masticatory mucosa, lining mucosa or specialized mucosa. In yet another embodiment, the compound is contained within a mouthwash, oral gel, oral spray or toothpaste.

The present disclosure also provides a method of treating or preventing a microbial infection in a mammal in need thereof, comprising administering to the mammal a compound having the following formula, a related compound or a pharmaceutically acceptable salt thereof:

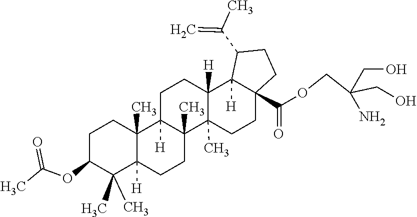

In one embodiment, the related compound has the following general formula:

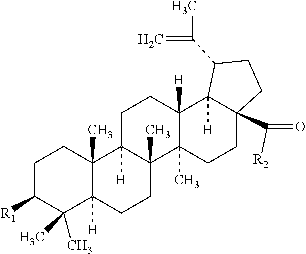

wherein R₁ represents a hydroxy group, an amino group, a protected hydroxy group or a protected amino group,

### and R₂=

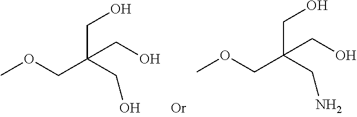

In another embodiment, R1 represents a hydroxy group, an amino group or one of the following protected hydroxy or amino groups:

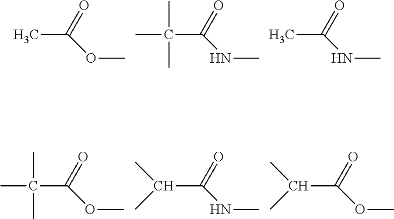

and R₂ is as defined above.

In one embodiment, the compound is formulated in a pharmaceutical composition. In another embodiment, the compound is administered topically, intramuscularly, intravenously, subcutaneously, orally or by inhalation. The method may further comprise administering a second compound to the mammal. In one embodiment, the pharmaceutical composition is a cream, ointment, gel, paste, solution, suspension, salve or aerosol. In another embodiment, the compound is administered to the oral cavity. In yet another embodiment, the mammal is a human. In another embodiment, the infection is caused by bacteria, fungi, protozoa or viruses. The bacterium may be a Gram negative or a Gram positive bacterium. In one embodiment, the second compound is an antibiotic. In another embodiment, the antibiotic is neomycin, bacitracin, polymyxin, mupirocin, erythromycin, azithromycin, penicillin, doxycycline, tetracycline, amoxicillin, ampicillin or vancomycin.

The present disclosure provides a cosmeceutical comprising a compound having formula (I), a related compound, or a salt thereof:

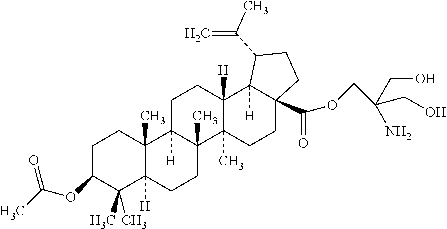

In one embodiment, the related compound has the following general formula:

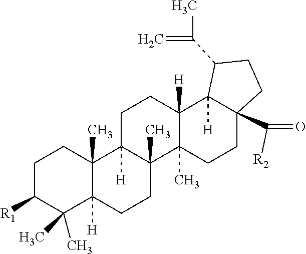

wherein R₁ represents a hydroxy group, an amino group, a protected hydroxy group or a protected amino group,

### and R₂=

In one embodiment, R1 represents a hydroxyl group, an amino group or one of the following protected hydroxyl or amino groups:

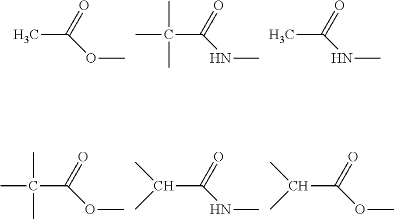

and R₂ is as defined above.

In another embodiment, the compound is formulated into a moisturizer, cream, lotion, toner perfume, lipstick, eye makeup, facial makeup, nail polish, powder, deodorant or sunscreen. The cosmetic may further comprise one or more biologically active agents. In another embodiment, the one or more agents are those listed in Table 1. In another embodiment, the cosmeceutical is a cream, ointment, gel, paste, solution, suspension, salve or aerosol.

The present disclosure also provides a method of improving the appearance and/or function of skin, comprising applying the cosmeceutical described above to the skin in an individual in need of such improvement. In one embodiment, the improved appearance is anti-aging, wrinkle reduction, improved/altered microcirculation, reduced redness, reduced inflammation, lightening of the skin, or reduced teleangiectasias.

### BRIEF DESCRIPTION OF THE DRAWINGS

FIGS. 1A-B show that the betulinic acid derivative NVX-207 was well tolerated after 2 weeks of application to a treatment site on human skin. The area within the circle on the right (red circle) was treated with a 1% topical solution of NVX-207 in 20% ethanol, 20% Tween® 80 in water. NVX-207 was applied topically once a day in a volume of 200 μl. The area within the circle on the left (black circle) was treated with the vehicle (20% ethanol, 20% Tween® 80 in water) as a control (FIG. 1A). The treatment site areas were then evaluated after two weeks for any adverse effects (FIG. 1B). No evidence of any side effects was found at the end of the treatment as well as 6 months thereafter.

FIGS. 2A-C show that topical application of NVX-207 effectively treated, i.e. improved a canine lesion clinically diagnosed as basal cell carcinoma. Differential diagnoses included virus induced lesions such as warts which are caused by papillomaviruses, and are thus an example of anti-microbial/anti-viral activity. A 1% topical solution of NVX-207 in 20% ethanol, 20% Tween® 80 was applied to an exophytically growing slightly bleeding (on contact) tumor on the nose of a dog using a NVX-207 soaked cotton bud (FIG. 3A) every second to third day for 7 weeks. Significant improvement with an about 90% reduction was observed after 3 weeks and bleeding stopped (FIG. 2B); except for a small prominent scar, the lesion had almost disappeared by week 7 (FIG. 2C). Twenty weeks after the end of treatment no evidence of re-growth was observed. Notably, treatment effects were observed in the absence of inflammation or infection.

FIGS. 3A-D show the effects of intratumoral treatment of NVX-207 in various spontaneously arising canine malignancies (before and after pictures). FIG. 3A shows an NVX-207 and cisplatin induced a complete remission in a dog with squamous cell carcinoma that was resistant against treatment by surgery, radiation or systemic chemotherapy. Complete remission was observed after intratumoral treatment once every three weeks for six weeks. FIGS. 3B-D show NVX-207 treatment effects in a canine mammary carcinoma and two separate cases of soft tissue sarcoma, respectively. Notably, NVX-207 therapy also completely prevented an often seen treatment—related inflammation. Microbial infections (with bacteria, fungi, viruses or protozoa) usually almost invariably accompany multiple injections under similar non-sterile conditions, because the coat of the animal cannot be efficiently sterilized. This in turn leads to unwanted treatment interruptions.

FIG. 4 shows the effects of NVX-207 on about 80% confluent as well as on about 30% confluent human endothelial (HUVEC), fibroblast and keratinocyte cell cultures. All cells were treated with 2.5 μM NVX-207. Percentage (means±S.E., n=3) of control-treated cells are given. Mean survival in human umbilical vein endothelial cells (HUVEC) at 80% confluency (black bar) was 90.3% (S.E.=0.9) versus 18.3% (S.E.=3.9) at 30% confluency (grey bar). Mean survival in NVX-207 treated fibroblasts at 80% confluency was 92% (S.E.=3.8) versus 3.8% mean survival (S.E.=0.4) at 30% confluency. In keratinocytes, mean survival (at 80% confluency) was 92.7% (S.E.=2.9) versus 17% (S.E.=4.3) in 30% confluent cell cultures. HUVECs, keratinocytes and fibroblast cells were obtained from Lonza GmbH (Wuppertal, Germany) and cultivated according to the manufacturer's instructions.

FIG. 5 shows the results of a safety study in pigs to evaluate the safety of increasing concentrations of topically administered NVX-207. Five pigs were used for evaluating the tolerability of topically applied NVX-207 at 0.5%, 1.0% and 2.0% in 20% ethanol/20 Tween® 80 in water. NVX-207, as well as vehicle controls, was applied to defined areas on the animals' backs once daily over two weeks. Skin areas were visually inspected every day. No macroscopic NVX-207 related skin irritations were observed during the course of the study. Results of blood chemistry and hematology did not reflect any systemic NVX-207-induced impact on blood status, mineral homoeostasis, and liver as well as kidney functions. No treatment—related histopathological changes were observed in biopsies of NVX-207 treated skin. Treated areas are marked by red circles. A, Vehicle control; B, 0.5% NVX-207; C, 1.0% NVX-207; D, 2.0% NVX-207.

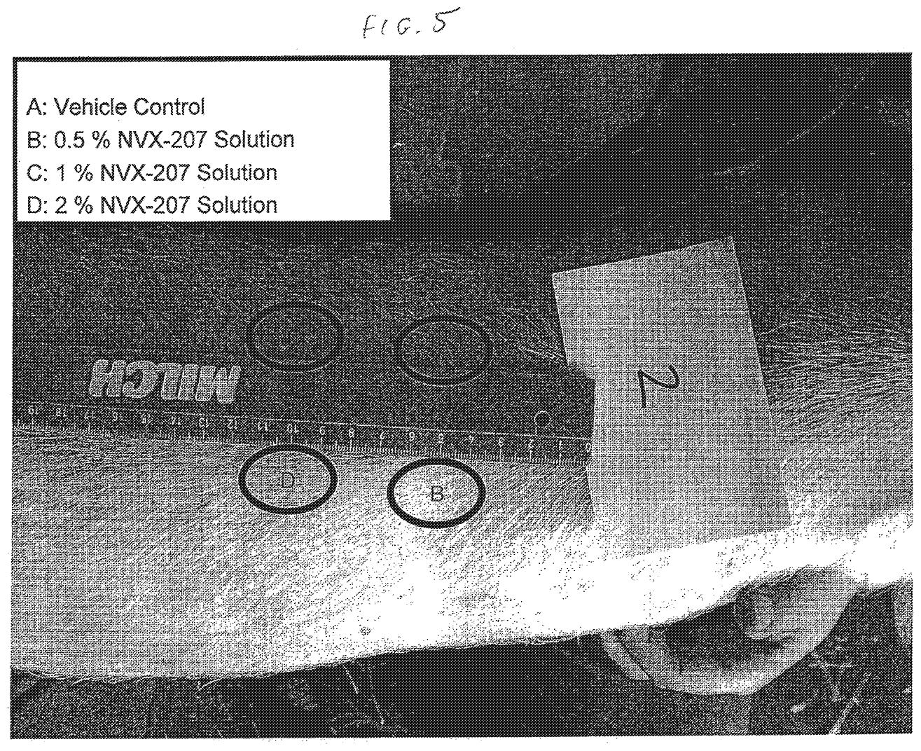

FIG. 6 shows the effects of NVX-207 against bacteria obtained from the skin surface of an individual, in either a cyclodextrin formulation (NVX-207 (at 10 mg/ml final concentration) was formulated in 2-hydroxypropyl-gamma-cyclodextrin (at 150 mg/ml final concentration) in water) (FIG. 6A), or a Tween 80/Ethanol formulation (FIG. 6B). 10 mm filter disks (numbered 1 to 4) were soaked in 10 μl of 10.0, 5.0, 2.5, and 1.25 mg/ml of NVX-207, respectively, and placed on LB-Agar plates on which a skin bacterial isolate had been plated. 15 ml of LB/Agar was used for pouring one plate. Control disks were soaked in 10 μl of the corresponding solvent and paced in the center of the plates. LB-Agar plates were then cultivated over night at 30° C. and bacterial growth was monitored by comparing growth inhibition zones around the soaked disks. A strong growth inhibitory effect was seen with NVX-207 formulated in cyclodextrin at 10 mg/ml. The distribution volume of NVX-207 in this experiment amounts to approximately 1 ml, representing a ˜100-fold dilution and a 100 μg/ml final concentration of NVX-207. The Tween/Ethanol formulation was much more effective, as strong inhibitory effects were seen at estimated concentrations of as low as 1.0 μg/ml NVX-207, assuming a distribution volume of this formulation of ˜2.5 ml in the LB-Agar plate.

FIGS. 7A-C show that topical application of NVX-207 effectively treated, i.e. improved, a human hyperkeratotic lesion with an inflammatory component and improved the appearance of the surrounding skin. The skin microcirculation appears improved, teleangiectasias appear less pronounced and redness appears reduced. A 1% topical solution of NVX-207 in 20% ethanol, 20% Tween® 80 was applied to a treatment resistant hyperkeratotic palpable lesion (FIG. 7A) once a day on days 1, 2, 3, 10, 11, 12 and 13. Significant improvement (better than 80% reduction) was observed after only one treatment on the next day (FIG. 7B), and additional improvement was observed after a total of six additional treatments applied within 14 days (FIG. 7C). After 14 days, the initial lesion was not palpable and no reddening, inflammation or any side effects of the skin was observed. A second identical but smaller lesion was treated on a different location (behind the right ear) with the same formulation twice a day for 2 days, and completely disappeared.

### DETAILED DESCRIPTION

The present disclosure relates to methods of treatment or prevention of infection by identifying a mammal in need of such treatment or prevention, and administering the betulinic acid derivative 3-O-acetylbetulinic acid-2-amino-3-hydroxy-2-hydroxymethylpropyl ester (herein referred to as NVX-207, (“the compound”)), related compounds or pharmaceutically acceptable salts thereof to the individual. The compound, related compound, or a pharmaceutically acceptable salt thereof can be used to treat or prevent bacterial, viral, fungal or protozoal infections. Related compounds are disclosed in U.S. Pat. No. 7,312,205, the entire contents of which are incorporated herein by reference. The synthesis and structure of NVX-207 and related compounds are described in U.S. Pat. No. 7,312,205, the entire contents of which are incorporated herein by reference. The compound may be formulated as a pharmaceutical composition, and may include one or more physiologically acceptable carriers or diluents.

The structure of NVX-207 is shown below:

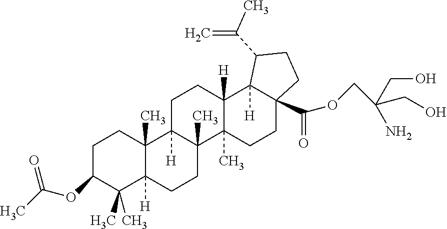

As used herein the term “a related compound” encompasses any of the betulinic acid derivatives disclosed in U.S. Pat. No. 7,312,205 which have the following general formula:

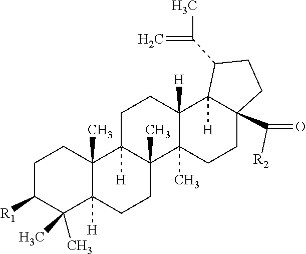

wherein R₁ represents a hydroxy group, an amino group, a protected hydroxy group or a protected amino group. Suitable protective groups are described in, for example, Chapters 2 and 7 of “Protective Groups in Organic Synthesis”, T. W. Greene and P. G. M. Wuts, 3^{rd} Edition, John Wiley & Sons, Inc. (1999), the disclosure of which is incorporated herein by reference,

### and R₂=

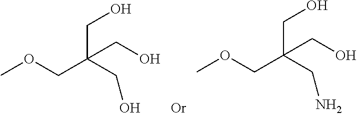

Other compounds related to NVX-207 having the general formula (I) as indicated above, suitable for use in the compositions and methods described herein are those in which R₁ represents a hydroxy group, an amino group or one of the following protected hydroxy or amino groups:

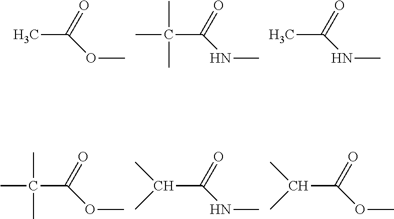

and R₂ is as defined above.

Infections which can be treated using the compounds and methods described herein include those of the skin and mucous membranes, including the oral cavity, ophthalmic infections, as well as systemic bacterial infections. NVX-207 can be used alone, or may be combined with one or more antibiotics conventionally used to treat bacterial infections, anti-viral agents used to treat anti-viral infections, anti-mycotics used to treat fungal infections, and/or anti-plasmodial compounds. Suitable antibiotics include neomycin, bacitracin, polymyxin, mupirocin, erythromycin, azithromycin, penicillin, doxycycline, tetracycline, amoxicillin, ampicillin, and vancomycin. Suitable anti-viral agents include acyclovir, interferon, efavirenz, amantadine, ganciclovir, imiquimod and ribavirin. Suitable antimycotics include Amphotericin B, nystatin, ketoconazole, miconazole, clotrimazole, itraconzaole, and natamycin. Suitable anti-protozoal agents include chloroquine, proguanil, metronidazole, tinidazole and doxycycline. The use of NVX-207 allows reduction of the required amount of the other compound(s), thus reducing the likelihood of side effects of the other compound(s) and/or reduces the likelihood that e.g. bacteria will become resistant to these conventionally used treatment modalities. Combinations of NVX-207 and related compounds with other antibiotics may also be helpful in the case of mixed infections, in case one singe agent does not covers the complete microbial spectrum of the bacterial infection.

NVX-207 and related compounds can be used to treat and/or prevent infection caused by both Gram positive and Gram negative bacteria, and thus to treat and/or prevent diseases caused by these agents. Gram positive bacteria include many well-known genera such as Bacillus, Listeria, Staphylococcus, Streptococcus, Enterococcus, and Clostridium. Medically relevant species of these bacteria include Listeria monocytogenes, the causative agent of listeriosis; Bacillus cereus (food poisoning), The definition of Gram positive bacteria has also been expanded to include the Mollicutes, bacteria like Mycoplasma that lack cell walls and cannot be Gram stained, but are derived from such forms. Actinobacteria are the other major group of Gram-positive bacteria. Gram negative bacteria include Escherichia coli, Salmonella, and other Enterobacteriaceae, Pseudomonas, Moraxella, Helicobacter, Stenotrophomonas, Bdellovibrio, acetic acid bacteria, Legionella and alpha-proteobacteria as Wolbachia and many others. Other notable groups of Gram-negative bacteria include the cyanobacteria, spirochaetes, green sulfur and green non-sulfur bacteria. Medically relevant Gram-negative cocci include three organisms, which cause a sexually transmitted disease (Neisseria gonorrhoeae), a meningitis (Neisseria meningitidis), and respiratory symptoms (Moraxella catarrhalis).

Medically relevant Gram-negative bacteria include a multitude of species. Some of them primarily cause respiratory problems (Hemophilus influenzae, Klebsiella pneumoniae, Legionella pneumophila, Pseudomonas aeruginosa), primarily urinary problems (Escherichia coli, Proteus mirabilis, Enterobacter cloacae, Serratia marcescens), and primarily gastrointestinal problems (Helicobacter pylori, Salmonella enteritidis, Salmonella typhi). Gram negative bacteria associated with nosocomial infections include Acinetobacter baumanii, which cause bacteremia, secondary meningitis, and ventilator-associated pneumonia in intensive care units of hospitals.

Many disorders and diseases are caused, in whole or in part, by bacterial infections. Any such disorder can be treated with NVX-207 or a related compound according to the methods described herein, including but not limited to, abscesses, acne, rosacea, telangiectasia, bacterial bronchitis, bacterial meningitis, third degree burns, blepharitis, conjunctivitis, sinusitis, Gonorrhea, Syphillis, psoriasis, mastitis and urinary tract infections, such as cystitis. The compound may also be administered to prevent any such bacterial infection in an individual prone to such infection. For example, the compound may be topically applied to a skin laceration to prevent bacterial infection, or may be systemically administered to treat a systemic bacterial infection.

NVX-207 and related compounds can be used to treat and/or prevent infection caused by viruses (e.g., herpes virus, papilloma virus, influenza virus, pox virus, hepatitis A virus, hepatitis B virus, hepatitis C virus, rhinovirus, coxsackie virus, parvovirus, HIV virus, and adenovirus), fungal agents (e.g., Candida spp., Trichosporon spp., Rhodotorula spp., Aspergillus spp, hyaline molds and dematiaceous molds) and protozoal agents (e.g., plasmodia, trypanosomes, Leishmania, Giardia intestinalis, Histomonas, Naegleria and Acanthamoebae.

Compared to betulinic acid, NVX-207 unexpectedly exhibits an over 20-fold increase in solubility in DMSO and an over 300-fold increase in solubility in ethanol. In addition, NVX-207, but not betulinic acid, can by formulated in cycodextrins, and re-dissolved in water as an active compound. Due to these outstanding chemical characteristics, NVX-207 can be applied topically with much higher efficacy as compared to e.g., betulinic acid.

After dissolving in ethanol or DMSO, or chemically similar solvents, NVX-207 or a related compound may be formulated with co-solvents such as Cremophor EL® and/or Tween® 80, in a similar manner described for taxanes. For example, the formulation approach for paclitaxel (TAXOL®; Bristol-Myers Squibb) was to use 50% Cremophor EL® and 50% ethanol. The pharmaceutical formulation of paclitaxel contains 30 mg paclitaxel dissolved in 5 ml of this (1:1, v/v) mixture. These and equivalent formulations allow for the application of NVX-207 in sufficiently high concentrations not only for topical, but also for intralesional and systemic (intravenous infusion) use. Solubility of NVX-207 in ethanol was determined to be at least 300 mg/ml and reached 250 mg/ml in DMSO. Solubility of betulinic acid in ethanol (<1 mg/ml) as well as in DMSO (10 mg/ml) is, by comparison, significantly lower. The solubility of NVX-207 in ethanol-Tween® 80 was evaluated. Two-hundred mg of NVX-207 was dissolved in 1 ml ethanol. The solution appeared to be clear and showed a light yellow color. Then an equal volume of Tween® 80 was added, to achieve a stock solution of 100 mg/ml of NVX-207. The stock solution was clear and yellow. Each stock solution was diluted first 1:1 (50 mg/ml NVX-207), then 1:5 (20 mg/ml NVX-207) and finally 1:10 (10 mg/ml NVX-207) with water. The final diluted solutions remained clear. NVX-207 therefore has, apart from its higher activity, significant advantages over betulinic acid, that include higher activity, better solubility in pharmaceutically suitable solvents, and better handling.

The term “pharmaceutical composition” refers to a mixture of NVX-207, a related compound, or a pharmaceutically acceptable salt thereof, with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable” defines a carrier or diluent that does not abrogate the biological activity and properties of the compound.

The term “pharmaceutically acceptable salt” refers to a formulation of a compound that does not cause significant irritation to an organism to which it is administered and does not abrogate the biological activity and properties of the compound. Pharmaceutical salts can be obtained by reacting a compound disclosed herein with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound of the disclosed herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glutamine, tris(hydroxymethyl)methylamine, and salts with amino acids such as arginine, lysine, and the like.

The term “metabolite” refers to a compound to which NVX-207 or a related compound is converted within the cells of a mammal. The pharmaceutical compositions disclosed herein may include a metabolite of NVX-207 or a related compound instead of NVX-207 or a related compound. The scope of the methods disclosed herein includes those instances where NVX-207 or a related compound is administered to the patient, yet the metabolite is the bioactive entity.

In a further aspect, the present disclosure relates to a method of treating a patient with a pharmaceutical composition as described herein.

The term “treating” or “treatment” does not necessarily mean total cure. Any alleviation of any undesired signs or symptoms of the disease or infection to any extent or the slowing down of the progress of the disease or infection can be considered treatment. Furthermore, treatment may include acts that may worsen the patient's overall feeling of well being or appearance.

The pharmaceutical compositions comprising NVX-207 or a related compound described herein can be administered to an animal, such as a mammal (e.g., dog, cat, horse, pig, cow, goat, sheep) or human per se, or in pharmaceutical compositions where they are mixed with other active ingredients, as in combination therapy, or suitable carriers or excipient(s). Techniques for formulation and administration of the compounds of the instant application can be found in “Remington's Pharmaceutical Sciences,” Mack Publishing Co., Easton, Pa., 18th edition, 1990.

Suitable routes of administration include, for example, oral, rectal, topical, transmucosal, oral mucosal or intestinal administration; parenteral delivery, including intralesional, intramuscular, subcutaneous, intravenous, intramedullary injections, as well as intrathecal, direct intraventricular, intraperitoneal, intranasal, or intraocular injections.

Furthermore, one can administer the drug in a targeted drug delivery system, for example, in a liposome coated with a tissue-specific antibody. The liposomes will be targeted to and taken up selectively by the organ.

The pharmaceutical compositions disclosed herein can be manufactured in a manner that is itself known, e.g., by means of conventional mixing, dissolving, granulating, dragee-making, levigating, emulsifying, encapsulating, entrapping or tabletting processes.

Pharmaceutical compositions for use in accordance with the present disclosure thus can be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients can be used as suitable and as understood in the art; e.g., in Remington's Pharmaceutical Sciences, above.

For injection, NVX-207 or a related compound can be dissolved in aqueous solutions after formulation with Tween® 80, Cremophor EL®, or similar detergents, preferably in physiologically compatible buffers such as Hanks's solution, Ringer's solution, or physiological saline buffer. Similar formulation techniques as those used for the taxanes can be utilized here. For transmucosal administration, penetrants appropriate to the barrier to be permeated are used in the formulation. Such penetrants are generally known in the art.

In addition, NVX-207 or a related compound can be dissolved in aqueous solutions after formulation in cyclodextrins.

For oral administration, NVX-207 or a related compound can be formulated readily by combining the active compound with pharmaceutically acceptable carriers well known in the art. Such carriers enable the compounds of the disclosure to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with the pharmaceutical combination of the disclosure, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

For topical administration, NVX-207 or a related compound can be formulated for administration to the epidermis as solutions, emulsions, ointments, gels, creams, pastes, salves, sunscreens, or lotions, shampoos, formulated in a topical or transdermal delivery system or formulated for ophthalmic use in an ophthalmically acceptable ointment or solution. The present compounds can also be applied in pure form, i.e., as liquids. However, it will generally be desirable to topically administer them to the skin as compositions or formulations, in combination with a dermatologically acceptable carrier, which may be a solid or a liquid. In one embodiment, the compound is administered to the oral cavity in which the compound contacts the oral mucosa, which is the mucous membrane epithelium of the mouth. The administration to the oral cavity can be with an oral rinse (e.g., mouthwash), oral gel, oral spray, oral dressing, or toothpaste comprising the compound. The compound can also be formulated for ophthalmic use, in an ophthalmically acceptable ointment or solution for treatment of eye infections, such as blepharitis and conjunctivitis.

A transdermal or topical delivery system can also involve a bandage or dressing designed to permit passage of a medicament to the skin or through the skin (by absorption therethrough) without preliminary puncture or abrasion of a living body. Transdermal drug delivery involves the delivery of drugs through the skin. Topical drug delivery systems apply formulations to the skin with the goal of localized application and minimal systemic circulation. With topical drug delivery systems, the active drug does not enter the bloodstream. Guidance for combining NVX-207 or a related compound with transdermal or topical delivery systems for application of a drug to the skin to achieve therapeutic effects are disclosed in detail in generally available references (Topical Drug Bioavailablity, Bioequivalence, and Penetration (eds. Vinod P. Shah and Howard I. Maibach, Plenum Press, 1993), and in Skin Barrier, Principles of Percutaneous Absorption (Hans Schaefer and Thomas E. Redelmeier, Karger Publ., 1996; Benson, Heather A. E., Transdermal Drug Delivery: Penetration Enhancement Techniques, Current Drug Delivery 2:23-33 (2005); Topical Drug Delivery Formulations edited by David W. Osborne and Anton H. Amann (available at http://chipsbooks.com/topdrug.htm); Hadgraft, Jonathan1, Lane, Majella E., Passive Transdermal Drug Delivery Systems: Recent Considerations and Advances, American Journal of Drug Delivery, Volume 4, Number 3, 2006 , pp. 153-160(8)).

Ointments and creams can, for example, be formulated with an aqueous or oily base with the addition of suitable thickening and/or gelling agents. Lotions can be formulated with an aqueous or oily base and will in general also contain one or more emulsifying agents, stabilizing agents, dispersing agents, suspending agents, thickening agents, or coloring agents.

In another embodiment, the compound is administered to the oral cavity for the chemoprevention or treatment of precancerous oral lesions (e.g., oral leucoplakia, erythroplakia and oral submucous fibrosis). In the oral cavity, the compound is delivered so as to contact the oral mucosa at the site(s) of precancerous lesions. The administration to the oral cavity may be with an oral rinse (e.g. mouthwash), oral gel, oral spray, oral paste, or toothpaste.

One preferred topical formulation comprises NVX-207 or a related compound and ethanol and/or DMSO, and one or more non-ionic detergents, including Cremophor EL® (polyethoxylated castor oil), Tween® 80 (polyoxyethylen-sorbitan-monooleate), Tween® 40 (polyoxyethylene (20)-sorbitan-monopalmitate), Triton® X-100 (polyethylenglycol-[4-(1,1,3,3-tetramethylbutyl)phenyl]-ether), Span® 20 (sorbitan monolaurate) and Span® 85 (sorbitan trioleate).

The combined amount of ethanol and/or DMSO can range from 0.1% to 70% (v/v). In one embodiment, about 20% (v/v) ethanol and/or DMSO is/are used. The total amount of nonionic detergent(s) can range from 0.1% to 80% (v/v). In one embodiment, about 20% (v/v) nonionic detergent is used.

The compound may be delivered to the oral cavity via dragee cores, which are provided with suitable coatings. For this purpose, concentrated sugar solutions can be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally, including sublingually, which include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of tablets or lozenges formulated in conventional manner.

For administration by inhalation, the compounds for use according to the present disclosure are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compounds can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions can be prepared by incorporating the active compound in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. Optionally, the suspension can also contain suitable stabilizers or agents which increase the solubility of the compounds to allow for the preparation of highly concentrated solutions.

Alternatively, the active ingredient can be in powder form for constitution with a suitable vehicle, e.g., sterile pyrogen-free water, before use.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

One example of a pharmaceutical carrier for the hydrophobic compounds disclosed herein is a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Tween® 80, and 65% w/v polyethylene glycol 300, made up to volume in absolute ethanol. Naturally, the proportions of a co-solvent system can be varied considerably without destroying its solubility and toxicity characteristics. Furthermore, the identity of the co-solvent components can be varied: for example, other low-toxicity nonpolar surfactants may be used instead Tween® 80; the fraction size of polyethylene glycol can be varied; other biocompatible polymers can replace polyethylene glycol, e.g., polyvinyl pyrrolidone; and other sugars or polysaccharides can substitute for dextrose.

Alternatively, other delivery systems for hydrophobic pharmaceutical compounds can be employed. Liposomes and emulsions are well known examples of delivery vehicles or carriers for hydrophobic drugs. Certain organic solvents such as dimethylsulfoxide also can be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days.

Many of the compounds used in the pharmaceutical combinations disclosed herein can be provided as salts with pharmaceutically compatible counterions. Pharmaceutically compatible salts can be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

Pharmaceutical compositions suitable for use in the present disclosure include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions of the present disclosure can be chosen by the individual physician in view of the patient's condition (See e.g., Fingl et al. 1975, in “The Pharmacological Basis of Therapeutics”, Ch. 1 p. 1). Typically, the dose range of the composition administered to the patient can be from about 0.5 to 1000 mg/kg of the patient's body weight. The dosage may be a single one or a series of two or more given in the course of one or more days, as is needed by the patient. Note that for almost all of the specific compounds mentioned in the present disclosure, human dosages for treatment of at least some condition have been established. Thus, in most instances, the present disclosure will use those same dosages, or dosages that are between about 0.1% and 500%, more preferably between about 25% and 250% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient can be, for example, a topical dose of between 0.01 mg and 10 mg of each ingredient, preferably between 1 mg and 10 mg. The daily dosage regimen for an adult human patient can be, for example, an oral dose of between 10 mg and 1000 mg of each ingredient, preferably between 10 mg and 500 mg, e.g. 25 to 500 mg or an intralesional or subcutaneous dose of each ingredient between 0.01 mg and 100 mg, preferably between 0.1 mg and 60 mg, e.g. 1 to 40 mg of each ingredient of the pharmaceutical compositions of the present disclosure or a pharmaceutically acceptable salt thereof calculated as the free base, the composition being administered, for example, 1 to 4 times per day. Alternatively the compositions of the disclosure can be administered by continuous intravenous infusion, preferably at a dose of each ingredient up to 400 mg per day. Thus, the total daily dosage by oral administration of each ingredient will typically be in the range 1 to 2500 mg and the total daily dosage by parenteral administration will typically be in the range 0.1 to 400 mg. Suitably the compounds can be formulated for continuous administration over a period of, for example, a week or more, or for months or years. In one embodiment, the formulations (as described above) for administration to the oral mucosa are administered every day, every other day, or every third day, one to three times per day for one week, two weeks, three weeks, one month, several months, one year or longer.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays can be used to determine plasma concentrations.

Dosage intervals can also be determined using MEC value. Compositions should be administered using a regimen that maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

Useful dosages of the compound can be determined by comparing its in vitro activity, and in vivo activity in animal models. Methods for the extrapolation of effective dosages in mice, and other animals, to humans are known to the art; for example, see U.S. Pat. No. 4,938,949.

Generally, the concentration of the compound in a liquid composition, such as a lotion, is from about 0.1-25 wt-%, preferably from about 0.5-10 wt-%. The concentration in a semi-solid or solid composition such as a gel or a powder is generally about 0.1-5 wt-%, preferably about 0.5-2.5 wt-%.

The amount of the compound, or an active pharmaceutically acceptable salt or derivative thereof, for use in the methods described herein will vary with the route of administration, the nature of the condition being treated and the age and condition of the patient and will be ultimately at the discretion of the attendant physician or clinician. In general, however, a suitable dose is in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound can be conveniently administered in unit dosage form; for example, containing 5 to 1000 mg, 10 to 750 mg, or, 50 to 500 mg of active ingredient per unit dosage form.

In one embodiment, the active ingredient is administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose can conveniently be presented in a single dose or as divided doses administered at appropriate intervals, for example, as two, three, four or more sub-doses per day. The sub-dose itself can be further divided, e.g., into a number of discrete loosely spaced administrations; such as multiple inhalations from an insufflator or by application of a plurality of drops into the eye.

The ability of a compound disclosed herein to act as an antibacterial agent can be determined using pharmacological models which are well known to the art, including the tests described in the examples below.

The compounds disclosed herein can also be useful as pharmacological tools for the further investigation of the mechanism of their antibacterial action.

The compositions can, if desired, be presented in a pack or dispenser device which may contain one or more unit dosage forms containing the active ingredient. The pack can, for example, comprise metal or plastic foil, such as a blister pack. The pack or dispenser device may be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions comprising a compound of the present disclosure formulated in a compatible pharmaceutical carrier can also be prepared, placed in an appropriate container, and labeled for treatment of an indicated condition.

The compositions described herein can also be used in the preparation of a medicament for treatment of any of the disorders described above.

One embodiment relates to a cosmeceutical comprising the betulinic acid derivative 3-O-acetylbetulinic acid-2-amino-3-hydroxy-2-hydroxymethylpropyl ester (herein referred to as NVX-207, or “the compound”) or a related compound as disclosed in U.S. Pat. No. 7,312,205, the entire contents of which are incorporated herein by reference, or a salt thereof. The synthesis and structure of NVX-207 and related compounds are described in U.S. Pat. No. 7,312,205. The compound, or related compounds, may be formulated as a cosmeceutical composition, and may include one or more physiologically acceptable carriers or diluents. NVX-207 has the structure shown below:

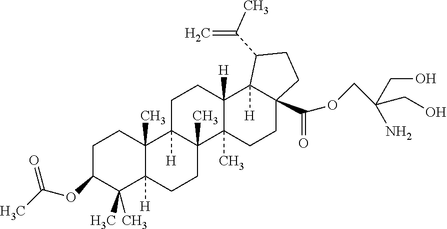

As used herein the term “a related compound” encompasses any of the betulinic acid derivatives disclosed in U.S. Pat. No. 7,312,205 which have the following general formula:

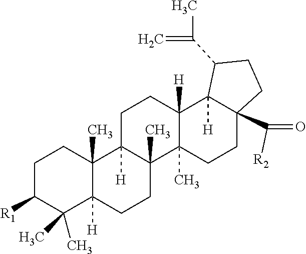

wherein R₁ represents a hydroxy group, an amino group, a protected hydroxy group or a protected amino group. Suitable protective groups are described in, for example, Chapters 2 and 7 of “Protective Groups in Organic Synthesis”, T. W. Greene and P. G. M. Wuts, 3^{rd}

Edition, John Wiley & Sons, Inc. (1999), the disclosure of which is incorporated herein by reference,

### and R₂=

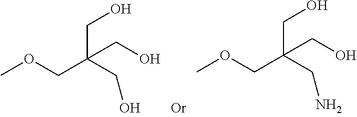

Other compounds related to NVX-207 having the general formula (I) as indicated above, suitable for use in the compositions and methods described herein are those in which R₁ represents a hydroxy group, an amino group or one of the following protected hydroxy or amino groups:

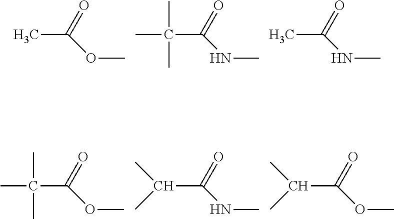

and R₂ is as defined above.

Another embodiment relates to a method of improving the biological function and/or appearance of the skin by topically applying a cosmeceutical comprising NVX-207, a related compound, or a salt thereof. These compositions improve the appearance, functioning and/or texture of the skin by, for example, one or more of the following: reducing the number and severity of wrinkles, reducing age spots and freckles, reducing skin damage, reducing redness, reducing teleangiectasias, improving and/or altering the microcirculation, altered keratinocyte differentiation, antibacterial effects, promoting lightening, and reducing inflammation, thus improving the appearance of aging, inflamed or damaged skin. Without wishing to be bound by any particular theory, promotion of skin lightening may involve antagonism of α-melanocyte stimulating hormone (α-MSH) and α-MSH induced cAMP generation, as well as other mechanisms. Thus, incorporation of NVX-207 or a related compound into a cosmeceutical composition has an overall anti-aging effect.

The term “cosmeceutical” encompasses any cosmetic composition having at least one biologically active ingredient that is applied to the skin, hair or mouth, including, but not limited to, a cleanser, moisturizer, cream, lotion, toner, perfume, lipstick, facial and eye makeup (e.g., foundation, powder, rouge, blush, mascara, eye liner, eye shadow, concealer), powders, deodorants, and sunscreens. Makeup compositions can be formulated, for example, as a foundation cream, powder, liquid or paste makeup base, which differ primarily in viscosity whereby beneficial effects are produced by application of the makeup composition to the skin.

In one embodiment, the cosmeceutical comprises one or more additional ingredients such as those provided in Table 1, which have the listed biological activity (Dureja et al., Indian J. Pharmacol., 37:155-159, 2005). For example, NVX-207 or a related compound, is combined with one or more anti-aging components such as a free radical scavenger, UV absorbing compound, moisturizing/rehydrating component or antioxidant component to produce an anti-aging moisturizer.

TABLE 1
Common cosmeceutical ingredients

| Ingredient | Purported action |
|---|---|
| Vitamins A, C and E | Antioxidant |
| α-Hydroxy acids (AHAs) | Exfoliates and improves circulation |
| β-Hydroxy acids (BHAs) | Antibacterial |
| Essential fatty acids | Smoothens, moisturizes and protects |
| Coenzyme Q10 | Cellular antioxidant |
| (Ubiquinone) | |
| Allatonin | Soothes |
| Aloe vera | Softens skin |
| Arnica | Astringent and soothes |
| Calendula | Soothes, softens, and promotes skin-cell |
| | formation |
| β-Bisabolol | Antiinflammatory, antibacterial, and calms |
| | irritated skin |
| Cucumber | Cools, refreshes, and tightens pores |
| Lupeol (dietary triterpene) | Antioxidant and skin conditioning agent |
| Ginkgo | Antioxidant that smoothes, rejuvenates, and |
| | promotes youthful appearance |
| Ivy | Stimulates circulation and helps other |
| | ingredients penetrate skin |
| Panthenol | Builds moisture and soothes irritation |
| Witch hazel | Tones |
| Green tea extract | Antioxidant |
| Neem oil limonoids | Antimicrobial |
| Pycnogenol | Anti-aging effect |
| α-Lipoic acids, | Potent free-radical scavengers and |
| Resveratrol, polydatins | antioxidant |
| Furfuryladenine | Improves hydration and texture of skin |
| Kinetin | Free-radical scavenger and antioxidant |
| Sodium hyaluronate | Lubricant between skin tissues and maintains |
| | natural moisture |
| β-Carotene | Minimizes lipid peroxidation and cellular |
| | antioxidant |
| Retinoic acid | Smoothes skin, promotes cell renewal and |
| | improves circulation to skin |
| Tetrahydrocurcuminoides | Antioxidant and antiaging |
| Centella | Skin conditioning agent, increases collagen |
| | production, improves texture and integrity of |
| | skin, and reduces appearance of stretch |
| | marks |
| Boswellia (triterpene) | Antiinflammatory and antiaging |
| Coriander seed oil | Antiinflammatory and antiirritant, skin- |
| | lightening properties |
| Turmeric oil | Antibacterial and antiinflammatory |
| Ingredient | Purported action |
| Coleus forskoflii oil | Antimicrobial, aromatherapy/perfumer |
| Arjunolic extract | Antioxidant and antiinflammatory |
| Ursolic acid | Antiinflammatory, collagen build-up |
| Oleanolic extract | Antioxidant, antifungal, improves texture, |
| | and integrity of skin |
| Rosemary extract | Antioxidant, antimicrobial, and |
| | antiinflammatory |
| Licorice extract | Skin whitening properties, antioxidant, |
| | antimicrobial, and anti-inflammatory |
| Horse chestnut extract | Supports blood circulation, wound healing |
| | effect, and anti-inflammatory |
| Betulin | α-MSH |

Other ingredients that are commonly used in the formulation of cosmeceuticals include the following:

UV blocking agents include, but are not limited to, inorganic pigments such as titanium dioxide, zinc oxide, p-aminobenzoic acid (PABA), cinoxate, diethanolamine p-methoxycinnamate, digalloyl trioleate, dioxybenzone, ethyl 4-[bis[hydroxypropyl]aminobenzoate, 2-ethyhexyl 2-cyano-3,3-diphenylacrylate, ethylhexyl p-methoxycinnamate, 2-ethyhexyl salicylate, glyceryl aminobenzoate, homosalate (3,3,5-trimethylcyclohexylsalicylate), lawsone (2-hydroxy-1,4naphthoquinone) with or without dihydroxyacetone, methyl anthranilate, oxybenzone, Padimate A, Padimate 0, 2-phenylbenzimidazole-5 -sulfonic acid, triethanolamine salicylate, red petroleum and sulisobenzone.

Antioxidants include, but are not limited to, retinoids and retinoic acid (e.g., (β-carotene), ascorbic acid, tocopherol acetate, magnesium ascorbyl phosphate, ascorbyl polypeptide, ascorbyl dipalmitate, licorice extract, mulberry extract, green tea extract, L-lysine, lauroylmethionin, superoxide dismutase, BHA, BHT, silymarine, extract of milk-thistle, ladies mantle extract and horsetail extract.

Free-radical scavengers include, but are not limited to, stabilized vitamin C compounds including, for example, ascorbyl palmitate and ascorbic acid polypeptide; stabilized forms of vitamin E compounds, including for example, dl-alpha-tocopherol acetate, protein bonded vitamin E (Tocopherol polypeptide); stabilized beta carotene compounds, and botanical extracts known to contain free radical scavengers, such as for example, ginkgo biloba and combinations thereof.

Moisturizing/rehydrating agents include, but are not limited to, D, L-panthenol, D-panthenol, vitamin A palmitate, vitamin E acetate, methylsilanetriol mannuronate, natural oils such as tallow oil, macadamia nut oil, borage oil, evening primrose oil, kukui nut oil, rice bran oil, tea tree oil, a medium chain fatty acid ester of glycerol, such as glycerol triheptanoate, glyceryl trioctanoate, glycerol trioctanoate, silicones, silicone derivatives and plant extracts containing combinations of botanical compounds such as flavanoids, phenolic compounds, cationic tannins, amino acids, saposids, and mineral salts, evening primrose oil, and phospholipid encapsulated vesicles, such as, for example, phospholipid encapsulated Vitamin E and phospholipid encapsulated mineral water.

Some hyperkeratotic lesions, such as those seen in actinic keratosis and psoriasis, have inflammatory components and are characterized by inflammation. As described in Examples 2 and 3 below, NVX-207, or a salt thereof, can be used to treat inflammatory disorders, and do not result in inflammation at the treatment site. Thus, in one embodiment, NVX-207, a related compound, or a salt thereof, is incorporated into a cosmeceutical which is then used to enhance the appearance of the skin by preventing or treating inflammation. Since NVX-207 and related compounds have anti-inflammatory properties, they soothe the skin, and prevent free radical-induced skin damage which contributes to wrinkles and aging of the skin.

The cosmeceuticals described herein can also contain one or more preservatives, emollients, anti-irritants, anti-inflammatory agents, healing agents or emulsifiers. Examples of calming, soothing and softening agents include Vitamin A palmitate; Phytelene Complex EGX 244, which is a botanical blend of extracts of calendula, chamomile, linden, cornflower, matricaria and hypericum; allantoin; dipotassium glycyrrhizinate; stearyl glycyrrhizinate; bisabolo((3-cyclohexene-1-methanol-.varies.,4-dimethyl-.varies.(4-methyl-3 -pentenyl)); squalane NF; cetyl ester wax; shea butter; orange roughy oil and hydrogenated phospholipids. Anti-irritant and anti-inflammatory agents include, but are not limited to, dipotassium glycyrrhizic cid, stearyl glycyrrhizic acid and bisabolol.

Emollients include, but are not limited to, hydrocarbon oils and waxes such as mineral oil, polyethylene and paraffin; triglyceride esters, lanolin and derivatives; ether-esters such as fatty acid esters of ethoxylated fatty alcohols; and fatty acids having 10 to 20 carbon atoms, such as lauric, myristic, oleic and stearic.

Emulsifiers include, but are not limited to, nonionic emulsifiers, such as cetyl dimethicone copolyol or dimethicone polyol, either alone or in combination with other emulsifiers, such as other non-ionic emulsifiers, sorbitan esters and its ethoxylates, methyl sucrose esters and its ethoxylates, fatty alcohol ethoxylates, wherein the fatty acid moiety contains less than 20 carbon atoms; and block copolymers of propylene oxide and ethylene oxide.

The synthesis and structure of NVX-207 and related compounds are described in U.S. Pat. No. 7,312,205, the entire contents of which are incorporated herein by reference. NVX-207 can be formulated as a cosmeceutical composition, and can include one or more components conventionally found in the particular cosmeceutical.

As noted above, the term “cosmeceutical” refers to any cosmetic applied to the skin which includes at least one biologically active ingredient. These cosmeceuticals include, but are not limited to, a moisturizer, cream, lotion, toner, perfume, lipstick, eye makeup (e.g., mascara), facial makeup, deodorants and sunscreens.

The term “biologically active ingredient” refers to any compound capable of exerting a biological effect, including but not limited to those listed in Table 1.

The term “salt” refers to a formulation of a compound obtained by reacting a compound disclosed herein with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Salts can also be obtained by reacting a compound disclosed herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glutamine, tris(hydroxymethyl)methylamine, and salts with amino acids such as arginine, lysine, and the like.

Techniques for formulation of cosmeceuticals may be found in, for example, Cosmeceuticals, edited by Zoe Diana Draelos, Elsevier Saunders, 2005.

Cosmeceutical formulations for topical administration include creams, pastes, gels, salves, ointments, suspensions, sprays, aerosols or lotions. In one embodiment, the compound is administered to the oral cavity in which the compound contacts the oral mucosa, which is the mucous membrane epithelium of the mouth. The oral mucosa may be divided into three categories: masticatory mucosa (keratinized stratified squamous epithelium, found on the dorsum of the tongue, hard palate and attached gingival), lining mucosa (non-keratinized stratified epithelium, found almost everywhere else in the oral cavity), and specialized mucosa (taste bud regions on the dorsum of the tongue). The administration to the oral cavity can be with an oral rinse (e.g., mouthwash), oral gel, oral spray or oral dressing comprising the compound.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the desired area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver NVX-207 to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful amounts of NVX-207 can be determined by one of ordinary skill in the art. Generally, the concentration of the compound in a liquid composition, such as a lotion, is from about 0.1-25 wt-%, preferably from about 0.5-10 wt-%. The concentration in a semi-solid or solid composition such as a gel or a powder is generally about 0.1-5 wt-%, preferably about 0.5-2.5 wt-%. The amount of the compound, or a related compound, or a salt thereof, for use in the cosmeceuticals and methods described herein will vary with the type of topical administration, the nature of the function and/or appearance to be improved, and the age and condition of the individual and will be ultimately at the discretion of the individual.

In general, however, a suitable dose is in the range of from about 0.00005 to about 1 mg/kg, e.g., from about 0.0010 to about 0.75 mg/kg of body weight per day, such as 0.0003 to about 0.50 mg per kilogram body weight of the recipient per day, preferably in the range of 0.0006 to 0.90 mg/kg/day, most preferably in the range of 0.0015 to 0.60 mg/kg/day.

### Example 1

### Preparation of NVX-207

NVX-207 was prepared as described in U.S. Pat. No. 7,312,205. The reaction scheme is shown below, in which IV is NVX-207.
1) Acetyl betulic acid-2-amino-3-hydroxy-2-hydroxymethyl propyl ester IV (Compound B)

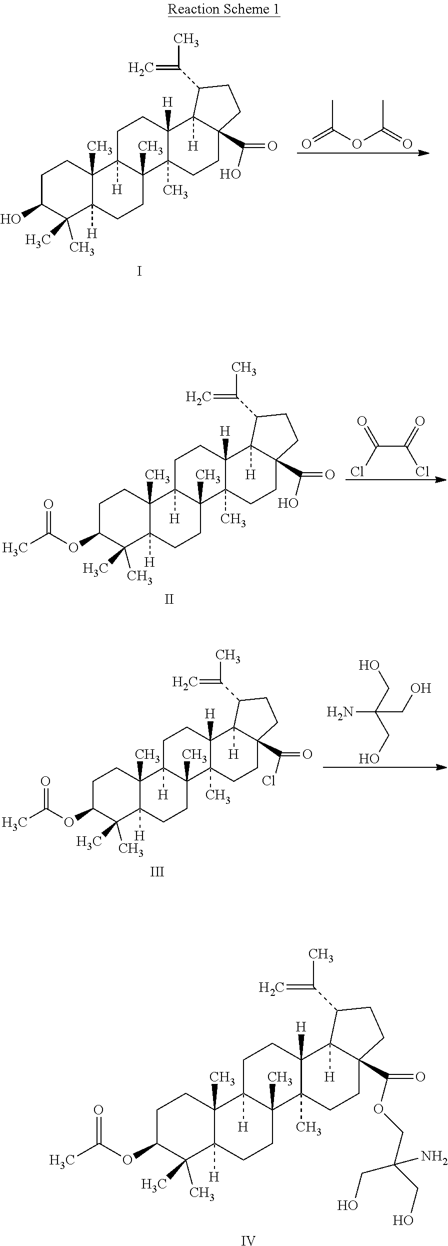

The synthesis of acetyl betulinic acid-2-amino-3-hydroxy-2-hydroxymethyl propyl ester IV is performed by departing from betulinic acid I via the intermediate stages of acetyl betulinic acid II and the respective acid chloride III, by reacting the acid chloride III with trishydroxymethylaminomethane.

a) Acetyl betulinic acid (MW 498.74) II

Two grams of betulinic acid I (MW 456.70) in 50 ml acetic anhydride are heated at reflux for 2 hours. After cooling, the reaction is poured into ice water under vigorous stirring, filtered, and the obtained solid is washed with water until the acetic acid smell has disappeared.

The solid is then heated at reflux in 70% ethanol for 4 hours under stirring.

After cooling, the reaction solution is filtered; the mother liquor is slightly concentrated, cooled in an ice bath and filtered once again. Yield: 86%; melting point: 290° C.

b) Acetyl betulinic acid chloride (MW 517.18) III

Two grams of acetyl betulinic acid II are provided in dry benzene and treated with a 10-fold excess of oxalyl chloride (3.4 ml). The reaction mixture is stirred for 8 hours under cooling, and the solvent as well as excess oxalyl chloride is subsequently evaporated on a rotary evaporator. In order to remove any oxalyl chloride residues, another 20 ml of benzene are added and again evaporated under vacuum.

c) Acetyl betulinic acid-2-amino-3-hydroxy-2-hydroxymethyl propyl ester (MW 601.86) IV (NVX-207) (compound B)

The acid chloride obtained from 1 g acetyl betulinic acid by the method according to b) is reacted without further purification. To this end, it is dissolved in 35 ml dioxan (dry), and tris(hydroxymethyl)-aminomethane is added in two-fold excess (0.004 mol, 0.5 g). After the addition of a spatula tip of DMAP and 3 drops of pyridine, the reaction mixture is stirred for 2 days at room temperature.

After this, the solids are filtered off; the solution is concentrated on the rotary evaporator and taken up in chloroform. This chloroform solution is washed free of pyridine with 1% hydrochloric acid, water and saturated saline solution several times. After drying over Na₂SO₄, the solvent is removed and the product is purified over a silica gel column or (and) chromatotron. A chloroform-methanol mixture at a ratio of 10:1 is used as an eluant. Yield: 20%, melting point: 156° C.
2) Acetyl betulic acid-N-(1,1-bis(hydroxymethyl)-2-hydroxyethyl)formamide (MW 601.86), (Compound C)

The synthesis of acetyl betulinic acid N-(1,1-bis(hydroxymethyl)-2-hydroxyethyl)formamide (compound C) is carried out in a manner analogous to reaction scheme 1, departing from betulic acid I via the intermediate stages of acetyl betulic acid II and the respective acid chloride III, by reacting the acid chloride III with tris(hydroxymethyl)aminomethane.

The acid chloride obtained from 1 g acetyl betulic acid (about 0.002 mol) according to the method of 1)b) is reacted without further purification. To this end, it is dissolved in 35 ml anhydrous dioxan, and an equimolar amount of tris-(hydroxymethyl)aminomethane (0.002 mol, 0.25 g) is added. After the addition of a spatula tip of DMAP and 3 drops of pyridine, the reaction mixture is heated to 80° C. for 8 hrs. After this, the reaction solution is concentrated on the rotary evaporator and the residue is taken up in chloroform. This chloroform solution is washed free of pyridine with 1% hydrochloric acid, water and saturated saline solution several times. After drying over Na₂SO₄, the solvent is removed and the product is purified over a silica gel column or (and) chromatotron. A chloroform-methanol mixture at a ratio of 10:1 was used as an eluant. Yield: 15%, mp: 184° C.

### Example 2

### NVX-207 is Well Tolerated by Human Skin

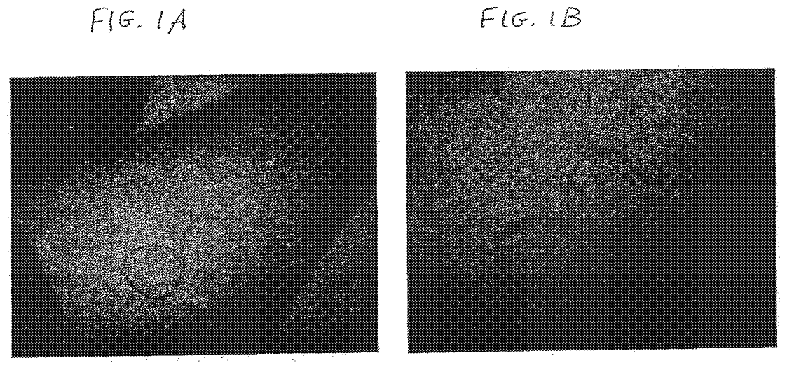

A 1% topical solution of NVX-207 (200 μl total volume in 20% ethanol, 20% Tween® 80) was applied to an area of skin on a human arm once a day for one week. A control solution (20% ethanol/20% Tween® 80 and 80% water) was applied to an area of skin adjacent to the area to which the NVX-207 solution was applied, also for one week. The results are shown in FIG. 1. The NVX-207-treated area (circle on right; FIG. 1B), and control-treated area (circle on left; FIG. 1A) exhibited no undesirable (toxic) effects. Thus, NVX-207, in contrast to 5-FU or Aldara®, is well-tolerated by human skin, and does not cause any adverse acute or late reactions.

### Example 3

### Treatment of Canine Basal Cell Carcinoma; Anti-Inflammatory Effects

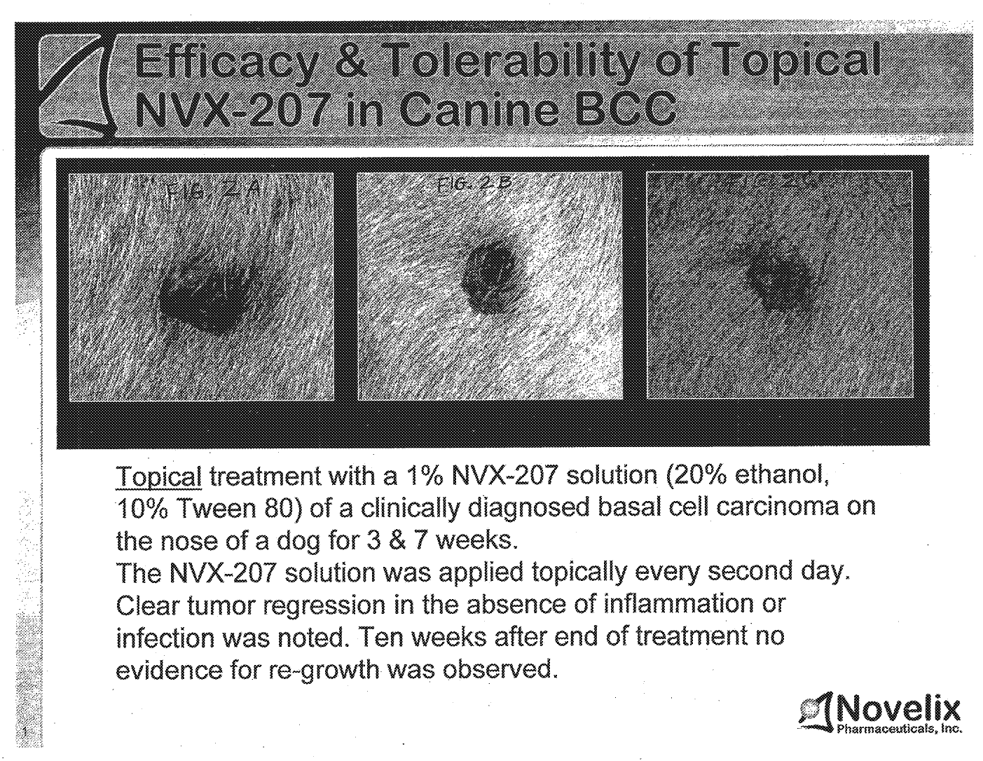

A 1% topical solution of NVX-207 in 20% ethanol, 20% Tween® 80 was applied to an exophytically growing slightly bleeding (on contact) tumor on the nose of a dog using a NVX-207 soaked cotton bud (FIG. 2A) every second to third day for 7 weeks. Significant improvement with an about 90% reduction was observed after 3 weeks and bleeding stopped (FIG. 2B); except for a small prominent scar, the lesion had almost disappeared by week 7 (FIG. 2C). Twenty weeks after the end of treatment no evidence of re-growth was observed. Notably, treatment effects were observed in the absence of inflammation or infection. This shows that topical application of NVX-207 effectively treated, i.e. improved a canine lesion clinically diagnosed as basal cell carcinoma and prevented infection at the treatment site.

### Example 4

### Treatment of Canine Tumors with NVX-207; Anti-Microbial Effects

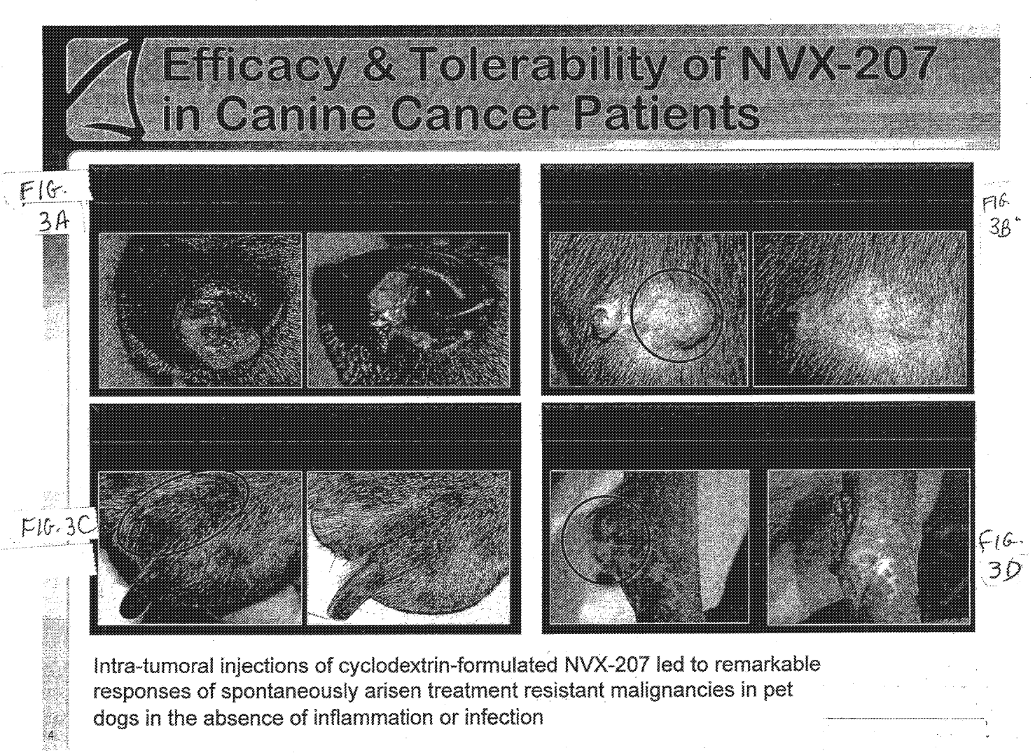

A study was performed using NVX-207 on a canine squamous cell carcinoma of the nasal plane. The carcinoma to which NVX-207 was applied did not respond to other modes of treatment, including surgical resection, radiation therapy and chemotherapy. Histology was confirmed by biopsy before treatment. Prior to each treatment, the dog was physically examined and routine blood counts and serum chemistry analyses were performed. Tumor measurements were obtained using callipers at each treatment. Tumor size was determined by the longest diameter of the target lesion or by calculating the mean of the longest diameters of the lesion treated. Tumor response was classified as stable (less than 50% change), partial—(decrease by >50%) or as a complete response if no palpable or visible evidence of tumor was available. Intralesional treatment of the patient comprised local infiltration of the local tumor, including 1 cm of the surrounding tissue, with NVX-207 in combination with cisplatin. The dog was treated by a combination of cisplatin (0.33 mg/ml final concentration)/NVX-207 (3.3 mg/ml final concentration) once every three weeks for 18 weeks, resulting in complete remission (FIG. 3A). Tumor recurrence was observed 18 months later. Thus, an unexpectedly long-lasting response was achieved with the combination of NVX-207/cisplatin in which cisplatin was used at one third the concentration than would have been used if cisplatin was administered alone. No systemic side effects were observed during the treatments, nor were there any signs of infection. No further complications were observed.

FIGS. 3B-D show NVX-207 treatment effects in a canine mammary carcinoma and two separate cases of soft tissue sarcoma, respectively. Notably, NVX-207 therapy also completely prevented an often seen treatment—related inflammation. Microbial infections (with bacteria, fungi, viruses or protozoa) usually almost invariably accompany multiple injections under similar non-sterile conditions, because the coat of the animal cannot be efficiently sterilized. This in turn leads to unwanted treatment interruptions.

Table 2 (below) shows the most important clinical parameters of four canine cancer patients treated intratumorally with NVX-207. Patients 1, 2, and 3 correspond to the dogs described above with squamous, soft tissue, and mammary carcinoma, respectively. Tumor size and tumor responses were determined as in described above. Intralesional treatment of the patients comprised local infiltration of the local tumor including 1 cm of the surrounding tissue—if applicable—with NVX-207 alone (for patients #2-#4) or in combination with cisplatin (patient #1, see also FIG. 3). Infiltration was carried out under either general or local anaesthesia, as appropriate. As a mono-substance, NVX-207 was used at 10 mg/ml concentrations formulated with the non-toxic solubilizer 2-hydroxypropyl-γ-cyclodextrin in water. Abbreviations used are: F, female; fs, female spayed; m, male; mc, male castrated; CR, complete remission of visible tumor, PR, partial remission, SD, stable disease. OS, overall survival time in months.

TABLE 2

| | | | Treatment | Tumor | |
|---|---|---|---|---|---|
| Patient | Age, gender | Tumor type | cycles | response | OS |
| #1 | 10, mc | Squamous cell | 12 | CR | 19 |
| | | carcinoma | | | |
| #2 | 11, fs | Soft tissue sarcoma | 48 | PR | 15 |
| #3 | 12, f | Mammary | 6 | SD | alive |
| | | carcinoma | | | |
| #4 | 5, m | Sweat gland | 6 | SD | alive |
| | | adenocarcinoma | | | |

Patient #1 was treated as described above in combination with cisplatin. No deleterious effects (e.g., redness, swelling, inflammation, infection) at the injection site were observed.

### Example 5

### Effects of NVX-207 on Primary Skin Target Cells (Endothelial Cells, Keratinocytes and Fibroblasts) of Psoriasis and Rosacea

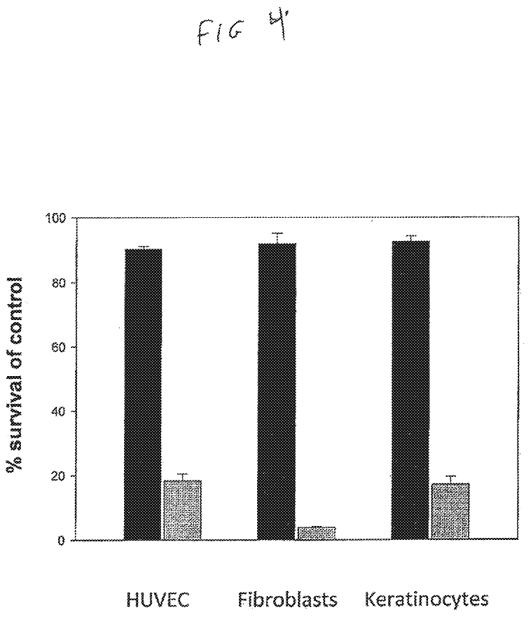

NVX-207 exhibited greatly enhanced anti-proliferative activity, compared to betulinic acid, against keratinocytes, the major cell type involved in psoriasis. The IC50 value of betulinic acid in human keratinocytes was recently determined to be ˜5.0 μg/ml (Tino Galgon al., Exp Dermatol. 14:736-43, 2005), while the IC50 of NVX-207 is ˜0.7 μg/ml. Consequently, the IC50 value of NVX-207 was almost 10-fold lower compared to betulinic acid on a molar basis. In addition, NVX-207 was also highly effective against two additional cell types important for psoriatic pathology, namely towards proliferating endothelial cells as well as fibroblasts. In sub-confluent (30% confluency) rapidly proliferating endothelial cells, a 2.5 μM concentration of NVX-207 was almost 5-fold more effective compared to nearly confluent endothelial cells (80% confluency) (FIG. 4). In a similar manner, NVX-207 was over 20-fold more active against proliferating human fibroblasts and almost 6-fold more active against keratinocytes (FIG. 4). Under normal physiological circumstances, the endothelium, as well as fibroblasts and keratinocytes of the tissue like normal skin, show relatively low turn-over and limited cell divisions to maintain tissue integrity. Accordingly, the compounds of the present disclosure and methods of using them achieved a preferential inhibition of growing vasculature and pathologically activated cellular compartments, an etiology which characterizes proliferative, highly active diseases such as psoriasis. Angiogenesis also plays a role in the pathogenesis of rosacea and a topical angiogenesis inhibitor (dobesilate) for treatment of rosacea has been described. Local inhibition of angiogenic factors function may prevent skin angiogenesis and inflammation in rosacea and other angiogenesis-dependent skin diseases in which a dense network of new vessels is produced and inflammatory cells are present. The preferential anti-endothelial effect of NVX-207 on proliferating cells may therefore be also of value in the treatment of this disease.

Keratinocytes at high confluence (e.g., 80%) mimic normal conditions in which these cells divide more slowly (fewer cell divisions per unit of time). In contrast, keratinocytes in psoriatic lesions divide more rapidly, as do keratinocytes cultured at lower confluency (e.g., 30%). Thus, the 30% confluency conditions were considered a model for psoriasis whereas the behavior of 80% confluent cells more closely reflects the conditions observed in normal skin. NVX-207 was much more effective on keratinocytes (as well as endothelial cells and fibroblasts) at 30% confluence when these cells are actively dividing, which supports the fact that this compound will effectively eliminate psoriatic keratinocytes, but will have little or no effect on normal keratinocytes, as has been observed by a lack of side effects in the normal skin in examples 2, 3 and 9.

### Example 6

### Topical NVX-207 Treatment is Well Tolerated in Pigs

A safety study in pigs was conducted to evaluate the potential toxicity of increasing concentrations of topically administered NVX-207. Five pigs with a mean final body mass of 16.4 kg were used for evaluating the tolerability of topically applied NVX-207. Increasing concentrations of NVX-207 (0.5%, 1.0% and 2.0% in 20% ethanol/20% polysorbate in 80% water) as well as vehicle controls were applied to defined areas on the animals' backs once daily over two weeks. Skin areas were visually inspected every day. Photographs were taken at three day intervals. At days 0, 7, and 14 blood samples were taken by puncture of the anterior vena cava. On day 14, pigs were anesthetized with ketamine and azaperon, and euthanized by intracardial application of T61®. No macroscopic NVX-207 related skin irritations were observed during the course of the study. Histopathological examination of skin biopsies taken on days 0 and 14 confirmed these findings. Results of blood chemistry and hematology did not reflect any systemic NVX-207-induced impact on blood status, mineral homoeostasis, and liver as well as kidney functions. In conclusion, topical administration of NVX-207 for two weeks was well tolerated in all animals tested. FIG. 2 is a photograph of pig #2 on treatment day 12. Treated areas are marked by red circles. A, Vehicle control; B, 0.5% NVX-207; C, 1.0% NVX-207; D, 2.0% NVX-207.

### Example 7

### Antibacterial Effect of NVX-207 on Skin Bacteria

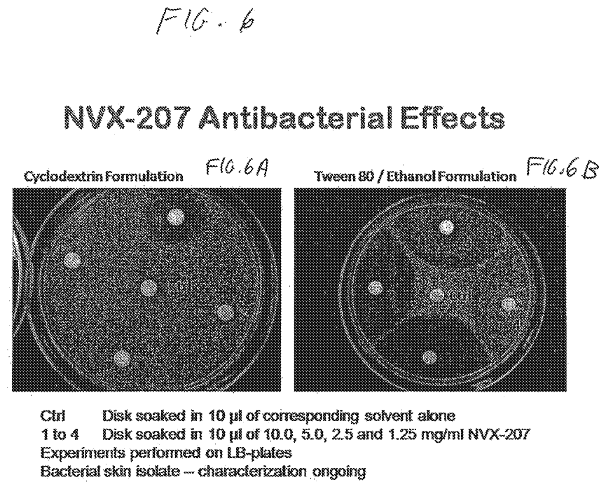

Antibacterial effects of NVX-207 were tested against bacteria obtained from the skin surface of an individual. NVX-207 was used either as a cyclodextrin formulation (NVX-207 (at 10 mg/ml final concentration) was formulated in 2-hydroxypropyl-gamma-cyclodextrin (at 150 mg/ml final concentration) in water) (FIG. 6A), or a Tween 80/Ethanol formulation (described in Example 2) (FIG. 6B). Filter disks 1 to 4 were soaked in 10 μl of 10.0, 5.0, 2.5, and 1.25 mg/ml of NVX-207, respectively, and placed on LB-agar plates (total volume of 15 ml LB-agar per plate) on which a skin bacterial isolate had been plated at low density. Control disks placed in the center of the plates were soaked in 10 μl of the corresponding solvent. LB-Agar plates were then cultivated over night at 30° C. and bacterial growth was examined after 18 hours. Growth inhibition was evident by a lack of a bacterial growth zone around the NVX-207 soaked filter disks. No growth inhibition was observed around the filter dishes soaked with vehicle alone. A growth inhibitory effect was seen with NVX-207 formulated in cyclodextrin at 10 mg/ml applied in a total volume of 10 μl. The distribution volume of NVX-207 in this experiment amounts to approximately 1 ml, representing a ˜100-fold dilution and a ˜100 μg/ml final concentration of NVX-207. The Tween/Ethanol formulation was much more effective, as strong inhibitory effects were seen at estimated concentrations of as low as 1 μg/ml NVX-207, assuming a distribution volume of ˜2.5 ml of NVX-207 in the LB-Agar plate volume in this experiment.

The results show that NVX-207 in the cyclodextrin formulation had a pronounced antibacterial effect at the highest concentration tested (10 mg/ml) (FIG. 6A). When the Tween 80/ethanol formulation was used, an antibacterial effect of NVX-207 was observed at all tested concentrations (FIG. 6B). Since bacterial growth is known to negatively impact the appearance of skin, reduction of such bacterial growth will improve the appearance of skin.

As noted above, strong evidence for an antibacterial activity of NVX-207 also comes from the canine cancer treatments. Dogs were treated over several months by repeated intralesional and peritumoral injections of NVX-207. In no case was any bacterial, viral, fungal or protozoal infection observed. This is highly unusual, since intralesional treatments under the same conditions are usually associated with a high incidence of serious local soft tissue infections leading to treatment interruptions. These observations therefore indicate an anti-infectious property of NVX-207 not only on bacteria growing on the human skin, but also in dogs.

### Example 8

### Treatment of Skin Infection

Ten individuals with minor skin bacterial infections are administered a topical ointment comprising NVX-207 twice a day for one week. Ten control individuals are administered vehicle (ointment not comprising NVX-207), and improvement is monitored. A significant, and more rapid, improvement in the individuals administered NVX-207 is observed compared to the individuals who are administered vehicle without NVX-207.

### Example 9

### Treatment of Acne

Ten individuals with moderate acne wash their faces twice a day for one month with a cleansing solution comprising NVX-207. Ten control individuals wash their faces twice a day with the control vehicle (solution not containing NVX-207), and improvement in the number and severity of blemishes is monitored. A significant, and more rapid, improvement in the individuals administered NVX-207 is observed compared to the individuals who are administered vehicle without NVX-207.

### Example 10

### Treatment of Conjunctivitis

Ten individuals with conjunctivitis are administered eye drops comprising NVX-207 twice a day for 10 days. Ten control individuals are administered the control vehicle (eye drops not containing NVX-207), and improvement is monitored. A significant, and more rapid, improvement in the individuals administered NVX-207 is observed compared to the individuals who are administered vehicle without NVX-207.

### Example 11

### Treatment of Oral Abscesses

Ten individuals with oral abscesses rinse with a solution comprising NVX-207 twice a day for one month. Ten control individuals rinse twice a day with the control vehicle (solution not containing NVX-207), and improvement in the infections is monitored. A significant, and more rapid, improvement in the individuals administered NVX-207 is observed compared to the individuals who are administered vehicle without NVX-207.

### Example 12

### Treatment of Bacterial Pneumonia

Ten individuals with early stage bacterial pneumonia are administered capsules comprising NVX-207 twice a day for two weeks. Ten control individuals are administered control tablets (tablets not containing NVX-207), and improvement is monitored. A significant, and more rapid, improvement in the individuals administered NVX-207 is observed compared to the individuals who are administered vehicle without NVX-207.

### Example 13

### Treatment of Rosacea

Ten individuals with rosacea are administered capsules comprising NVX-207 twice a day for two weeks. Ten control individuals are administered control tablets (tablets not containing NVX-207), and improvement is monitored. A significant, and more rapid, improvement in the individuals administered NVX-207 is observed compared to the individuals who are administered vehicle without NVX-207.

### Example 14

### Treatment of Human Hyperkeratotic Lesion and Improved Appearance of Surrounding Skin

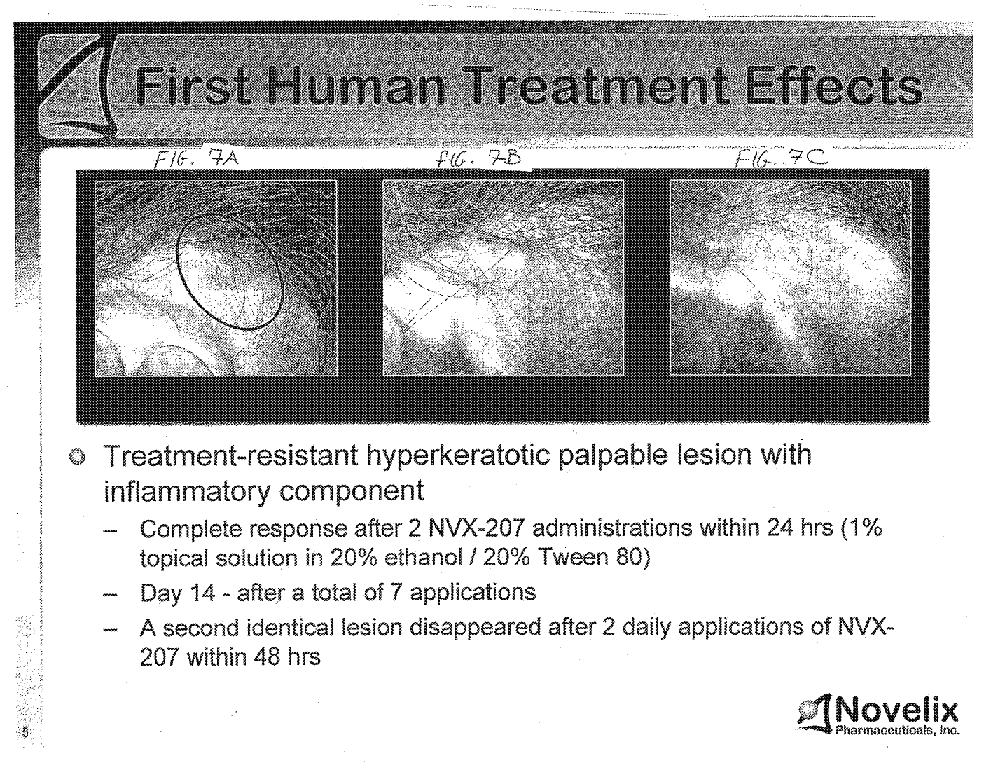

A 1% topical solution of NVX-207 (20% ethanol, 20% Tween® 80 and 80% water) was applied to a treatment resistant hyperkeratotic palpable lesion with inflammatory components. The skin before treatment is shown in FIG. 7A. Significant improvement was observed after one day (FIG. 7B), and the lesion had disappeared after 14 days (FIG. 7C). A second, identical lesion was treated with the same formulation twice a day for 2 days, and disappeared. No deleterious (toxic) effects on the treated areas (e.g., inflammation, redness, swelling) were observed. In the surrounding skin areas, skin microcirculation appears improved, teleangiectasias appear less pronounced and redness appears reduced.

### Example 15

### Skin Care Gel

The ingredients of the gel are listed in Table 3.

TABLE 3

| Ingredient | Amount (%, w/w) |
|---|---|
| NVX-207 | 1% |
| Cyclopentasiloxane (and) dimethicone | 70.44% |
| crosspolymer (and) cyclohexasiloxane | |
| Cyclomethicone | 19.52% |
| Propylene glycol | 3.64% |
| Soluble collagen | 0.49% |
| hydrolyzed silk protein | 0.72% |
| Glycerin | 2.98% |
| Preservatives | 1.02% |

The gel in this example is prepared according to the following procedure. The mixture of cyclopentasiloxane, dimethicone crosspolymer, cyclohexasiloxane (e.g., Silicone Elastomer Blend) is added to cyclomethicone and stirred in a manufacturing vessel. Propylene glycol, soluble collagen, hydrolyzed silk protein, glycerine, NVX-207 and preservatives are mixed in a separate vessel, and then added into the manufacturing vessel with continuous moderate stirring. Care is taken with stirring to avoid too much aeration.

The gel can be applied to the skin once daily, or as desired, to improve the overall appearance of the skin.

### Example 16

### Skin Cleansing Lotion

The ingredients listed in Table 4, and are combined to form a skin cleansing lotion.

TABLE 4

| Ingredient | Amount (%, w/w) |
|---|---|
| Water | 69.95% |
| NVX-207 | 1% |
| Sodium lauryl sulphate | 4.96% |
| Stearic acid | 3.18% |
| Decyl oleate | 2.28% |
| Lauramide DEA | 1.25% |
| Glyceryl stearate S.E. | 2.47% |
| Cetyl alcohol | 1.98% |
| Propylene glycol | 2.37% |
| Triethanolamine | 0.24% |
| Phospholipids | 0.82% |
| 1,3-butylene glycol | 0.69% |
| Cholesterol | 0.36% |
| Polysorbate-80 | 8.11% |
| Diazolidinyl urea | 0.29% |
| Methyl paraben | 0.11% |
| Propyl paraben | 0.05% |

The lotion can be used to cleanse the skin once daily, or as desired.

### Example 17

### Skin Toner Solution

The ingredients listed in Table 5 are combined to form a skin toner solution.

TABLE 5

| Ingredient | Amount (%, w/w) |
|---|---|
| NVX-207 | 1% |
| Sodium chloride | 2.13% |
| Potassium chloride | 0.48% |
| Potassium bromide | 0.54% |
| Magnesium chloride | 2.02% |
| Calcium chloride | 2.06% |
| Glycerin | 1.02% |
| Water | 91.54% |
| Preservatives | 0.21% |

The toner solution is prepared according to the following procedure. Sodium chloride, potassium chloride and potassium bromide are dissolved thoroughly into purified water by stirring. Magnesium chloride and calcium chloride are then added in a consecutive order while stirring until each one is totally dissolved. Glycerin, NVX-207 and the preservatives are then added. Before filling, the solution is passed through a 0.2-.mu.m filter to remove undissolved particles or other impurities.

The toner can be applied to the skin once daily after cleansing, or as desired.

### Example 18

### Skin Care Cream

| Ingredient | Amount (% w/w) |
|---|---|
| NVX-207 | 1% |
| Pentaerythrityl tetracaprylate/tetracaprate | 5.2% |
| Emulsifying wax | 3.9% |
| Behentrimonium methosulfate (and) | 3.15% |
| cetearyl alcohol | |
| Mineral oil | 0.88% |
| Soybean oil | 1.06% |
| Lanolin alcohol | 0.51% |
| Water | 81.72% |
| Calcium chloride | 0.22% |
| Glycerin | 1.9% |
| Preservatives | 0.93% |
| Fragrance | q.s. |

The cream in this example is prepared according to the following procedure. NVX-207, pentaerythrityl tetracaprylate/tetracaprate, emulsifying wax, behentrimonium methosulfate and cetearyl alcohol, mineral oil, soybean oil and lanolin alcohol are added to a manufacturing vessel, and the vessel is stirred and heated to 75° C. Calcium chloride is dissolved in water, and glycerin is then added. The aqueous solution was heated to 75° C., and added to the manufacturing vessel with quick stirring, resulting in a monophasic emulsion. The vessel is cooled to 40° C. When the desired temperature is reached, preservatives and fragrance are added and mixed thoroughly in the vessel.

The cream can be applied to the skin once daily, or as desired.

### Example 19

### Facial Makeup Composition

| Ingredient | Amount |
|---|---|
| NVX-207 | 0.25 g |
| Mixture of oxyethylenated oxypropylenated | 9 g |
| 9 g polymethylcetyl (dimethyl) (methyl)- | |
| siloxane, polyglyceryl-4 isostearate and hexyl | |
| laurate | |
| Tristearin mixture | 0.5 g |
| D₅/D₆ cyclomethicone | 25 g |
| Diphenyl dimethicone | 6 g |
| Isododecane | 4.55 g |
| Heccorite | 4 g |
| Particulate phase prepared by milling a 10 g | 10 g |
| BEF II plastic sheet sold by 3M Company | |
| Stabilized, partially neutralized vinyl | 20 g |
| acetate/vinyl p-tert butylbenzoate/crotonic | |
| acid copolymer in aqueous dispersion | |
| Diisopropyl adipate | 1 g |
| Water | q.s. to 100 g |

The pigment is predispersed in a portion of the cyclomethicone. The remaining oil is homogenized with the surfactants at 40-50° C., and the mixture is allowed to cool. The pigment and the modified hectorite, the latter having been pre-swollen in a small amount of isododecane, are added to the mixture. The entire aqueous phase is added to the above lipophilic phase, first with slow stirring, and then with vigorous stirring for 10 minutes. The copolymer and the diisopropyl adipate are then added with slow stirring.

The makeup formulation is applied to the skin as desired.

### Example 20

### Eye Shadow Composition

| Ingredient | Amount |
|---|---|
| NVX-207 | 0.5 g |
| Elastomeric crosslinked | 40 g |
| polydimethylsiloxane particles in an aqueous | |
| dispersion comprising 63% of crosslinked | |
| polymer (BY 29-119, Dow Corning) | |
| Glycerol | 5 g |
| Butylene glycol | 5 g |
| Goniochromatic liquid-crystal pigments | 20 g |
| (Helicone ® HC Scarabeus, Wacker) | |
| Mica-titanium oxide (Flamenco Blue from | 15 g |
| Engelhard) | |
| Pigments (black iron oxide) | 5 g |
| Nylon powder (Orgasol ® 2002 extra D NAT | 9 g |
| COS from ATOFINA) | |
| Preservative | 1 g |

The ingredients are mixed, and the composition is either sieved to give a loose powder, or is packed in a dish by pressing to give a compact product. The powder is applied to the eye area as desired.

Thus, the compounds and methods described herein provide for the effective treatment and/or prevention of a variety of infections. Additionally, the cosmeceutical preparations described herein, comprising the betulinic acid derivative 3-O-acetylbetulinic acid-2-amino-3-hydroxy-2-hydroxymethylpropyl ester and related compounds, can be used to influence the appearance and/or biological function of the skin by topical application of these preparations.

It will be understood by those of skill in the art that numerous and various modifications can be made without departing from the spirit of the present disclosure. Therefore, it should be clearly understood that the forms of the present disclosure described herein are illustrative only and are not intended to limit the scope of the present disclosure.

All documents and other information sources cited above are hereby incorporated in their entirety by reference.

What is claimed is:

1. A method of treating or preventing an infection in a mammal in need thereof, comprising identifying a mammal in need of such treatment, and administering an effective amount of a compound having the following formula, a related compound, or a pharmaceutically acceptable salt thereof, to said mammal:

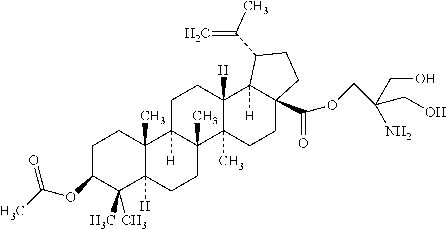

2. The method of claim 1, wherein said related compound has the following general formula: 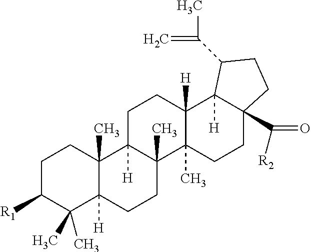
wherein R₁ represents a hydroxy group, an amino group, a protected hydroxy group or a protected amino group,
and R₂=

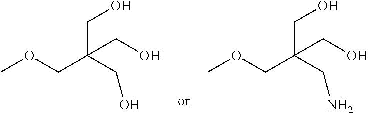

3. The method of claim 2, wherein wherein R₁ represents a hydroxy group, an amino group or one of the following protected hydroxy or amino groups: 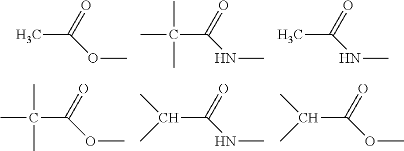
and R₂ is as defined above.

4. The method of claim 1, wherein said compound is administered topically.

5. The method of claim 4, wherein said compound is formulated as a lotion, ointment, gel, cream or paste.

6. The method of claim 4 wherein the compound is formulated in a transdermal delivery system.

7. The method of claim 1, wherein said compound is administered to the oral cavity.

8. The method of claim 7, wherein said compound is contained within a mouthwash, oral gel, oral spray or toothpaste.

9. The method of claim 1, wherein said compound is administered intravenously, intramuscularly, subcutaneously or via aerosol.

10. The method of claim 1, wherein said mammal is a human.

11. The method of claim 1, wherein said infection is a bacterial, viral, fungal or protozoal infection.

12. A method of treating or preventing an oral lesion resulting from an infection in a mammal in need thereof, comprising identifying a mammal in need of such treatment, and administering to the oral mucosa of said mammal an effective oral lesion-treating or preventing amount of a compound having the following formula, a related compound, or pharmaceutically acceptable salt thereof:

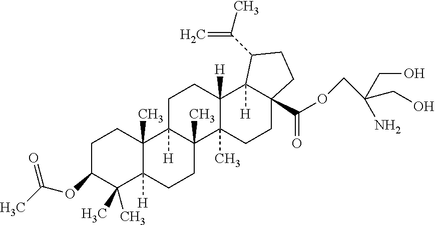

13. The method of claim 12, wherein said related compound has the following general formula: 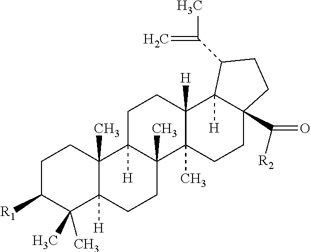
wherein R₁ represents a hydroxy group, an amino group, a protected hydroxy group or a protected amino group,
and R₂=

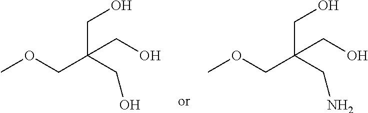

14. The method of claim 13, wherein R₁ represents a hydroxy group, an amino group or one of the following protected hydroxy or amino groups: 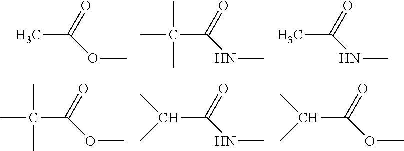
and R₂ is as defined above.

15. The method of claim 12, wherein said mammal is a human.

16. The method of claim 12, wherein said oral mucosa is masticatory mucosa, lining mucosa or specialized mucosa.

17. The method of claim 12 wherein said compound is contained within a mouthwash, oral gel, oral paste, oral spray or toothpaste.

18. The method of claim 12, wherein said oral lesion results from a bacterial, viral, fungal or protozoal infection.

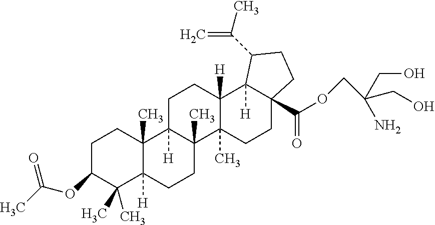

19. A method of treating or preventing a bacterial infection in a mammal in need thereof, comprising administering to the mammal a compound having the following formula, or a pharmaceutically acceptable salt thereof.

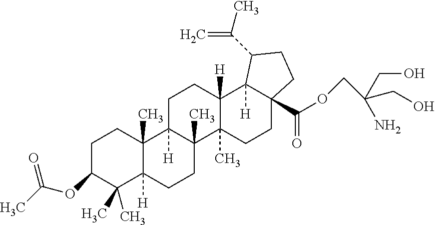

20. The method of claim 19, wherein said related compound has the following general formula: 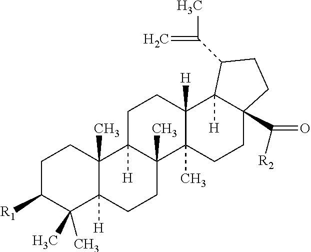
wherein R₁ represents a hydroxy group, an amino group, a protected hydroxy group or a protected amino group,
and R₂=

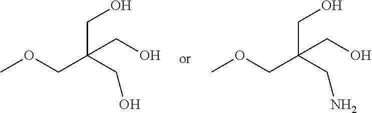

21. The method of claim 19, wherein R₁ represents a hydroxy group, an amino group or one of the following protected hydroxy or amino groups: 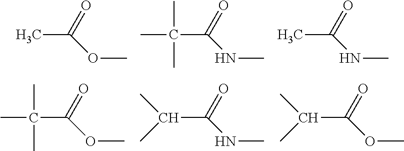
and R₂ is as defined above.

22. The method of claim 19 wherein said compound is formulated in a pharmaceutical composition.

23. The method of claims 19, wherein said compound is administered topically, intramuscularly, intravenously, subcutaneously, orally or by inhalation.

24. The method of claims 19, further comprising administering a second compound to said mammal.

25. The method of claim 19, wherein said pharmaceutical composition is a cream, ointment, gel, paste, solution, suspension, salve or aerosol.

26. The method of claim 19, wherein said compound is administered to the oral cavity.

27. The method of claim 19, wherein said mammal is a human.

28. The method of claim 19, wherein said infection is caused by a Gram negative bacterium.

29. The method of claim 19, wherein said infection is caused by a Gram positive bacterium.

30. The method of claim 24, wherein said second compound is an antibiotic.

31. The method of claim 30, wherein said antibiotic is selected from the group consisting of: neomycin, bacitracin, polymyxin, mupirocin, erythromycin, azithromycin, penicillin, doxycycline, tetracycline, amoxicillin, ampicillin and vancomycin.

32. A cosmeceutical comprising a compound having the following structure, a related compound, or a pharmaceutically acceptable salt thereof:

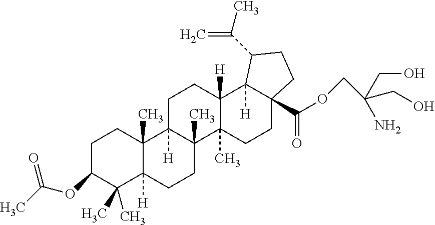

33. The cosmeceutical of claim 32, wherein said related compound has the following general formula: 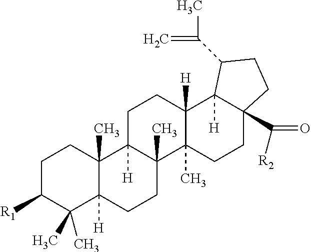
wherein R₁ represents a hydroxy group, an amino group, a protected hydroxy group or a protected amino group,
and R₂=

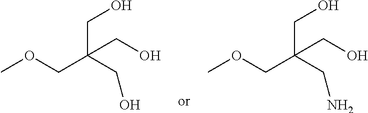

34. The cosmeceutical of claim 33, wherein wherein R₁ represents a hydroxy group, an amino group or one of the following protected hydroxy or amino groups: 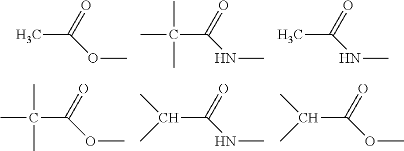
and R₂ is as defined above.

35. The cosmeceutical of claim 32 wherein said compound is formulated into a cosmetic selected from the group consisting of a moisturizer, cream, lotion, toner perfume, lipstick, eye makeup, facial makeup, powder, sunscreen and deodorant.

36. The cosmeceutical of claim 32, wherein said cosmeceutical further comprises one or more biologically active agents.

37. The cosmeceutical of claim 32, wherein said one or more agents is selected from the group consisting of the agents listed in Table 1.

38. The cosmeceutical of claim 32, wherein said cosmeceutical is a cream, ointment, gel, paste, solution, suspension, salve or aerosol.

39. A method of improving the appearance and/or function of skin, comprising applying the cosmeceutical of claim 32 to the skin of an individual in need of such improvement.

40. The method of claim 39, wherein said improved appearance is selected from the group consisting of anti-aging, wrinkle reduction, improved/altered microcirculation, reduced redness, reduced inflammation, lightening of the skin, and reduced teleangiectasias.
